(19) United States
(12) Patent Application Publication
(10) Pub. No.: US 2013/0289102 A1
(43) Pub. Date: Oct. 31, 2013

(54) AMYRIS ALCOHOL AND ITS ESTER FOR TREATING ACNE

(71) Applicants: Chandra Ulagaraj Singh, San Antonio, TX (US); Jagaveerabhadra Rao Nulu, Austin, TX (US)

(72) Inventors: Chandra Ulagaraj Singh, San Antonio, TX (US); Jagaveerabhadra Rao Nulu, Austin, TX (US)

(73) Assignee: Trinity Laboratories, Inc., San Antonio, TX (US)

(21) Appl. No.: 13/996,636

(22) PCT Filed: Dec. 22, 2011
(86) PCT No.: PCT/US2011/066751

§ 371 Date: Jul. 2, 2013

Related U.S. Application Data
(60) Provisional application No. 61/425,916, filed on Dec. 22, 2010.

(51) Int. Cl. A61K 31/22; A61K 31/12; A61K 31/352; A61K 31/045

(52) U.S. Cl. CPC A61K 31/22; A61K 31/045; A61K 31/12; A61K 31/352
USPC 514/450; 514/510; 514/729; 514/454; 514/453; 514/679

(57) ABSTRACT

The present invention relates to formulations comprising amyris alcohol or an ester of amyris alcohol or combinations thereof to treat acne in humans. These formulations can further comprise a phytoestrogen such as glabridin and miroestrol, an antioxidant such as tetrahydrocurcumin and naturally occurring peroxides such as artemisinin and dihydroartemisinin and combinations thereof for improved effectiveness in treating acne.

### CROSS REFERENCE TO RELATED APPLICATIONS

This application claims the benefit of U.S. Appl. No. 61/425,916, filed Dec. 22, 2010. The content of the aforesaid application is relied upon and incorporated by reference in its entirety.

### FIELD OF THE INVENTION

The field of the invention relates generally to therapeutics. More specifically, the field of the invention relates to treatments for acne.

### BACKGROUND OF THE INVENTION

There are approximately 45 million people who suffer from acne in America alone (http://www.aad.org/media-resources/stats-and-facts/conditions/acne). The disease is so common in youth at their puberty that it often has been termed physiological. Although acne stops appearing for most people by the age of 25, some people, the majority of them are women, experience the disease well into their adult life. This “adult acne” differs from teenage acne in location and that it tends to be more inflammatory with fewer comedones. As the human concern for facial beauty continues to receive heightened marketing attention, the cure for various forms of acne has received much attention, as evidenced by the number of patents and patent applications that have appeared recently. From 1975 to January 2003, there were over 9000 patents issued by the U.S. Patent Office that had some reference to acne. From these data, it would become obvious that a suitable solution to this problem has eluded past efforts.

The acne process results from a cascade of events. First, at puberty a spike in androgen production heralds an increase in sebum production and begins the hyperkeratinization process causing microcomedones and sebum blockade. With this blockage, the number of resident follicular flora increases dramatically. These bacteria produce inflammatory products, which permeate through thin walls of dilated sebum-filled duct. Once in the perifollicular dermis, they trigger the body's own immune defenses (both acute and granulaomatous) to produce the characteristic inflammatory papules, pustules and nodules characteristic of inflammatory acne.

Thus, there are four factors that are believed to be the contributors of acne: (1) Increased sebum production; (2) Comedo formation, in which the follicular infundibulum hypercornifies, hyperkeratinizes, and hypodesquamates; (3) Colonization of the follicule by anaerobic Propionibacterium, mainly P. acnes; and (4) The host's inflammatory response. The above four factors are interrelated to each other. Sebum is comedogenic and causes inflammation by itself. Propionibacterium acnes is a relatively slow growing, (typically) obligate anaerobe gram positive bacterium that is linked to the skin condition acne.

The Propionibacterium has high lipolytic activity and liberates free fatty acids from sebum lipids. The free fatty acids have been shown to cause marked inflammation. The microorganisms also produce other extracellular enzymes such as proteases and hyaluronidases, and chemotactic factors, which may be important in the inflammatory process. It would thus be advantageous to provide relief from all of the above four principal causes of acne.

Topical products used to remove comedones are known as comedolytics, the most effective being tretinoin, marketed as a prescription product (Retin A) and by several generic companies (http://www.drugs.com/pro/tretinoin.html). Tretinoin or all-trans retinoic acid is the naturally occurring metabolite of Vitamin A. Tretinoin increases epidermal cell turnover, thus causing comedolysis and most importantly prevents the formation of new keratinous plugs. Applications of tretinoin are normally once a day at bedtime. Dryness, stinging and redness sometimes accompany the applications. Importantly, improvement is usually not seen for 6-8 weeks. Adapalene 0.1% (Differin) is a topical retinoid like tretinoin. Available by prescription only, the gel is usually applied once nightly. Side effects include frequent scaling, burning, redness and dryness. Improvement is delayed and is not evident for 4-8 weeks. Sodium sulfacetamide 10%/sulfur 5% (Sulfacet-R) is also available by prescription only. It is a lotion with antibacterial and comedolytic action. As with tretinoin, improvement is seen in 4-8 weeks. Salicylic acid 2% is an over the counter product that exhibits mild comedolytic activity.

The only products that have anti-sebum activity are estrogens and 13 cis-retinoic acid (isotretinoin) and these must be used systemically to be effective (Strauss, J., et al., (2007); Guidelines of care for acne vulgaris management, Journal of the American Academy of Dermatology 56 (4): 651-63). Isotretinoin (Accutane) is a metabolite of Vitamin A available by prescription only. Isotretinoin is used to treat only severe cystic or conglobate acne. Because of its teratogenic properties, birth defects can occur. Isotretinoin is a powerful drug and can elevate triglycerides, total cholesterol and decrease high-density lipoproteins (HDL). Other side effects include dry skin, dry eyes, itching, headaches, nosebleed, and photosensitivity. It is generally taken for 4-5 months to see improvement. Recently, one brand of oral contraceptive has been approved for the treatment of acne for patients who request birth control.

A number of topical and systemic agents are used to lower the number of bacteria that colonize the follicular duct. These include benzoyl peroxide (BP), BP 5%/erythromycin 3% (Benzamycin). BP has antibacterial activity and drying effects and is available over the counter or by prescription. Moreover, it has been recently reported that benzoyl peroxide seems to induce free radical production that can produce skin changes that qualitatively resemble ultraviolet B damage, e.g., increases in epidermal thickness, and deleterious changes in elastin and glycosaminoglycans content (Ibbotson, S H., et al., J. Inves. Derm., 1999, 112: 933-938). In addition, Benzoyl peroxide is highly reactive, and is thus difficult to stabilize in practical compositions. BP is applied once or twice daily for 1-2 months. BP can produce erythema and peeling of skin. BP is often tried first for both non-inflammatory and mild inflammatory acne. Other topical antibiotics include clindamycin and erythromycin. These are used as solutions, lotions or gels by prescription only. Usually they are applied once or twice daily and results are seen in 1-2 months. Azelaic acid 20% (Azelex) also has mild antibacterial effects. Systemic antibiotics include tetracycline and its analogs, which are used in low doses for years or until the end of the acne prone years. Most patients with mild inflammatory acne receive a combination of topical antibiotics and tretinoin or other retinoid. Bacterial resistance does occur so antibiotics may be changed or BP is substituted since resistance does not occur with BP. More severe acne requires systemic antibiotics and topical retinoid. The most severe must receive oral isotretinoin for 4-5 months.

There are no drugs that directly affect the inflammatory acne. The retinoids do have some anti-inflammatory properties, but these are poorly described. Topical steroid and even systemic steroids have been used to abort a severe flare of fulminant acne, but these are limited uses because of the side effects. Benzoyl peroxide gels are sometimes used as first aid on acne lesions. These function as a “drawing poultice”, but data supporting this use is not available.

The treatment for acne centers on opening the pore, killing P. acnes, reducing sebum production and regulating inflammatory responses. Retinoids are the agents to reduce sebum production and open the pore. As a topical agent, Differin (adapalene) or Retin-A (tretinoin) is used for mild and moderate acne. Isotretinoin, an oral drug, is very effective but reserved for the severe and resistant acne because of its teratogenicity, hepatotoxicity, elevating triglyceride level and other side effects.

For topical applications, the Food & Drug Administration (FDA) has approved the following ingredients for marketing topical acne products in the USA (Code of Federal Regulations, 21 CFR 333.310); (1) Resorcinol (2%, in combination only); (2) Resorcinol monoacetate (3%, in combination only); (3) Salicylic acid 0.5 to 2 percent, and (4) Sulfur 3 to 10 percent.

Salicylic acid has been used to treat acne for some time. Salicylic acid dries the skin, which helps in acne management, but it also causes skin irritation in perilesional skin areas of acne patients, especially patients with sensitive skin, and in some cases the erythema is extreme. Salicylic acid is also pH-sensitive, as in neutralized forms, such as sodium salicylate or triethanolamine salicylate, there is a loss of efficacy due to poor bioavailability. In free acid form, salicylic acid is absorbed rapidly and transported into bloodstream. This is the reason for its irritation-causing problems.

Topical and oral antibiotics (especially tetracycline, erythromycin, and clindamycin) are sometimes prescribed for patients with inflammatory papules and pustules, but, in addition to the undesirability of antibiotic overuse in general, which can lead to enhanced susceptibility to infection, disadvantages to such treatments include phototoxicity and interactions with other medications. Other factors that play a role in exacerbating acne, including oil-based cosmetics and some drugs (e.g., androgenic hormones, high-progestin birth control pills, systemic corticosteroids, and iodide- and bromide-containing agents) are often minimized during acne treatment. Besides the side effects of the antimicrobial agents, development of resistant microorganisms has become an important issue nowadays. The number of patients harboring resistant P. acnes has been shown to be growing. For this reason, it would be advantageous to exclude antibiotics and antibacterial agents from topical preparations for acne.

For efficacious topical treatments, it would thus be advantageous to include the following six-prong provisions to control fundamental elements that can provide control of both acne and rosacea in a single composition: (1) Control of excess sebum production; (2) Control of undesirable bacteria and mites; (3) Control of inflammation; (4) Enhanced desquamation of follicular infundibulum cells; (5) Reduction of irritation from anti-acne and anti-rosacea compositions themselves; and (6) An enhancement of the topical bioavailability of anti-acne and anti-rosacea compositions.

Since the resistance to bacteria is becoming a problem, it would be advantageous to control bacteria without using an antibacterial agent. Also, salicylic acid is being one of the most favored and inexpensive ingredients to control acne, albeit its irritation causing side effects, it would be advantageous to devise methodologies to increase both topical bioavailability and anti-acne efficacy of salicylic acid with a reduction in its irritation causing side effects.

The literature abounds with acne and rosacea treatments. For example, about 1,000 medical papers have been published on this subject. From these data, it would become obvious that a suitable solution to acne problems has not yet been found. A discussion of some of the various patents and patent applications follows.

US Patent Application Pub. No.: 20100069495 (Graeber) discloses the administration of adapalene in a topical medicament to a patient so as to sustain its biological response in the treatment of acne vulgaris, wherein the administration pattern of the topical medicament comprises topically applying onto the afflicted skin area a therapeutically effective amount of adapalene at least once every two days for at least 6 months.

US Patent Application Pub. No.: 20090246156 (Kunin) discloses the compositions for anti-acne sunscreen. The sunscreen composition has the ability to treat and prevent acne in addition to screen both UVA and UVB radiation. In particular, the sunscreen composition includes a sunscreen base, at least one UVA deactivator, at least one UVB deactivator, and at least one anti-acne agent. The UVA deactivator may be avobenzone and the UVB deactivator may be selected from one of the following oxybenzone, octisalate, octyl methoxycinnamate, or a mixture thereof.

US Patent Application Pub. No.: 20090186809 (Hentsch) discloses a surprising therapeutic beneficial use for the topical application of valproic acid as a single agent therapy for patients suffering from mild to moderate acne vulgaris. Topically applied VPA has a clinical efficacy comparable to that of the marketed standard medication for this indication, isotretinoin. Furthermore, topically applied VPA is on average well to very well tolerated. The invention relates to the topical medical use of VPA for the treatment of acne vulgaris and comprises the topical application of VPA or of a pharmaceutically acceptable salt thereof.

US Patent Application Pub. No.: 20090209604 (Zhang) discloses aqueous topical compositions containing a combination of benzoyl peroxide, at least one dicarboxylic acid having 6 to 12 carbon atoms, and at least one vitamin B₃ compound in aqueous vehicles containing at least one 1,2-alkanediol having from 5 to 7 carbon atoms. The compositions are suitable for treatment of acne. Also described are methods for topically delivering a medicament to a human patient, in need of the medicament by topically administering to the patient the compositions of the present invention.

US Patent Application Pub. No.: 20080176908 (McAnally) discloses a method for the treatment of acne comprising the administration, to a patient afflicted therewith, of a effective amount of a squalene monooxygenase inhibitor such as tolnaftate, naftifine, terbinafine, butenafine or ciclopirox. An advantage of the present invention relates to the surprisingly speedy onset of effectiveness in relieving acne symptoms. The compound may be administered orally or topically.

US Patent Application Pub. No.: 20080014289 (Li) discloses a vehicle and method is for treating and/or preventing skin disorders such as acne vulgaris and which exfoliates healthy skin by a topical application. The vehicle is a formulation incorporating hypohalous acid in a suitable pharmaceutical compound. The topical application comprises administering and scrubbing a therapeutically effective amount of hypohalous acid in the vehicle. The hypohalous acid may be hypochlorous acid, hypobromous acid, or hypoiodious acid. The suitable pharmaceutical vehicles include water, solutions, cleansers, lotion, cream, paper facial masks, and gels. The scrubbing action on the skin is exerted by a mechanic tool, such as hand, cloth towel, sponge, brush, and spraying device. Methods of preparing and compounding the vehicle for topical application and of its use are also set forth.

US Patent Application Pub. No.: 20080153916 (Pisano) discloses the use of a substance belonging to the class of atypical retinoids for the topical treatment of acne is described. In particular the effects on an animal model for this skin disease of a pharmaceutical or cosmetic compound for topical use containing adamantyl methoxydiphenyl propenoic acid formulated in a gel are demonstrated.

US Patent Application Pub. No.: 20070207115 (Liegeois) discloses a system consisting of tea tree oil used alone or in combination with conventional over-the-counter strength anti-acne agents. A system is created whereby Tea tree oil is used in combination with other anti-acne therapies, particularly benzoyl peroxide. Subsequent steps utilize conventional anti-acne treatments.

US Patent Application Pub. No.: 20050271750 (Lee) discloses a topical formulation for preventing or treating acne, in particular a topical formulation for preventing or treating acne through the antimicrobial activity of the formulation against acne-causing bacteria, Propionibacterium acnes, inhibition of excess production of sebum by inhibition of excess production of sebum by inhibition of 5α-reductase, inhibition of comedo, keratolysis and anti-inflammatory action, which comprises extract obtained from at least one oriental medicine selected from the group consisting of Cavalia gladiata, Biota orientalis and Coptis chinensis.

US Patent Application Pub. No.: 20050008684 (Herdeis) discloses treatment of a skin disease selected from acne, rosacea, atopical dermatitis, and various ulcers, by topically applying a suitable pharmaceutical or cosmetic composition containing an effective amount of taurolidine, taurultam or a mixture containing both.

US Patent Application Pub. No.: 20040156873 (Gupta) discloses an invention relating to acne and rosacea compositions by a six-prong synergistic combination treatment strategy that includes (1) control of excess sebum production, (2) control of undesirable bacteria or mites, (3) control of inflammation, (4) enhanced desquamation of follicular infundibulum cells, (5) reduction of irritation from anti-acne or rosacea compositions themselves, and (6) enhancement of the topical bioavailability of anti-acne and rosacea compositions. This is achieved by a synergistic combination of commonly utilized topical anti-acne and rosacea ingredients with a topical bioavailability enhancement composition, which results in enhanced anti-acne and rosacea action from such ingredients. Moreover, additional inclusion of an anti-inflammatory composition, and also a vascular micro-circulation enhancement composition, further results in synergistic superior anti-acne and rosacea benefits from such compositions.

US Patent Application Pub. No.: 20030072777 (Maes) discloses a topical combinatorial composition comprising an anti-acne effective amount of an adhesion blocking component comprising at least a polysaccharide, a sebum reducing component comprising at least a pygeum extract, an anti-inflammatory component comprising at least a hoelen mushroom extract, an anti-irritant component, and a sclareolide component in a cosmetic or pharmaceutically acceptable vehicle

US Patent Application Pub. No.: 20070269537 (Shyam) discloses certain extracts of Artemisia annua plant, both in their crude and refined forms, and certain refined forms of Artemisia annua plant extracts composed substantially of Artemisinin and its analogs, which are chemically classified as sesquiterpenes with an endo-peroxide group. These are suitable for compositions comprising topical application, and for the treatment or improvement of skin condition including acne, rosacea, topical wounds, age spots, wrinkles, excess facial oil, and darkened skin.

U.S. Patent Application Pub. No.: 20030021855 (Perricone) discloses acne prevention by the topical application of compositions containing an alkanolamine such as dimethylaminoethanol, in combination with tyrosine and a sulfur ingredient such as lipoic acid or glutathione. Such alkanolamines have strong amine odor that is objectionable to consumers for application on face. Moreover, several such alkanolamines have a high pH that can cause irritation.

U.S. Patent Application Pub. No.: 20030021816 (Kang) discloses an immunosuppressant compound, a second active ingredient selected from the group consisting of comedolytics, antibacterials, anti-inflammatory, retinoids, glucocorticoids, and mixtures thereof, and a dermatologically acceptable carrier for acne treatment. Such immunosuppresants are not readily available for common use.

U.S. Patent Application Pub. No.: 20020192298 (Burrell) relates to the use of antimicrobial metals, preferably silver for the treatment of an acne. It is preferred that the use of any antimicrobial agents for acne treatment be minimized or eliminated due to development of resistant bacteria.

U.S. Patent Application Pub. No.: 20020172672 (Sieberg) is directed to the use of serine proteases, either alone or in combination with a retinoid compound in a pharmaceutical or cosmetic composition for acne treatment. Such enzyme preparations can cause serious skin allergy in some humans.

U.S. Patent Application Pub. No.: 20020155180 (Goodman) discloses treatment of acne that comprises topically applying an effective amount of a saw palmetto berry extract and one or more constituents that enhance penetration of the extract into hair follicle sebaceous glands. This disclosure is specific to one ingredient, hence of limited application.

U.S. Patent Application Pub. No.: 20020151527 (Wiegand) discloses a method for reducing the number and severity of acne lesions comprising administering a sensory regimen to down regulate the activity of the hypothalamus-pituitary-adrenal axis, in combination with the administration of a topical anti-acne composition comprising an anti-acne agent selected from salicylic acid, sulfur, lactic acid, glycolic acid, pyruvic acid, urea, resorcinol, N-acetylcysteine, retinoic acid, benzoyl peroxide, octopirox, triclosan, azelaic acid, phenoxyethanol, phenoxypropanol, flavinoids, derivatives thereof, and mixtures thereof. The problems of salicylic acid irritation and low topical bioavailability and the use of antibacterials are still not eliminated by Wiegand. U.S. Patent Application Pub. No.: 20010056071 (Pelicchia) discloses the application of antioxidant resveratrol for acne treatment.

U.S. Patent Application Pub. Nos: 20020172719, 20020054918, and 20020041901 (Murad) disclose pharmaceutical or cosmetic composition and methods for the cleansing of skin to facilitate the prevention, treatment, and management of skin conditions that include rosacea and acne by a composition that includes a hydroxy acid or tannic acid to exfoliate a portion of the skin, stabilized hydrogen peroxide to facilitate cleansing of the skin, and an antimicrobial agent to inhibit or reduce microorganisms on the skin. Since the overuse of antimicrobial agents can cause further problems, as mentioned earlier, Murad inventions are thus of limited application, or even to be possibly avoided for any long-term rosacea and acne treatment regimen.

U.S. Pat. No. 6,451,773 (Oester) discloses a combination of chitosan with azelaic acid, benzoyl peroxide, retinoic acid, salicylic acid, or mixtures thereof, for the treatment of acne. Chitosan is used as a film-forming agent for topical application of other active ingredients for better adhesion to skin surface. While topical bioavailability is enhanced, the skin irritation and other problems of salicylic acid and azelaic acid use are not reduced.

U.S. Pat. No. 6,440,994 (Sanders) discloses acne treatment using a mixture of antihistamines and anti-inflammatory agents. This does not provide a multifaceted treatment objective. U.S. Pat. No. 6,436,417 (Singh) discloses solubilized forms of salicylic acid for acne treatment. Such solubilized forms absorb more quickly, reaching bloodstream at a faster rate. Both the topical anti-acne efficacy may be lower and skin irritation may be higher for such compositions. U.S. Pat. No. 6,433,024 (Popp et al.) discloses topical anti-acne compositions based on benzoyl peroxide, an alpha hydroxy acid, a moisturizer, an isosorbide and a detergent. These compositions contain several skin irritating ingredients.

U.S. Pat. No. 6,365,623 (Perricone) discloses one preferred embodiment that contains a combination of lipoic acid, an α-hydroxy acid, and dimethylaminoalcohol. Lipoic acid is also claimed to cure rosacea (U.S. Pat. No. 6,472,432; Perricone), U.S. Pat. No. 6,262,117 (Sefton) discloses acne treatment based on a combination of benzoyl peroxide and azelaic acid. The poor stability of benzoyl peroxide and the skin irritation of either benzoyl peroxide or azelaic acid are still unsolved in Sefton disclosure. U.S. Pat. No. 6,168,798 (O'Halloran et al.) discloses an alcoholic solution of salicylic acid and salicylates for acne treatment. The rapid absorption of such clear solutions into skin would reduce the topical bioavailability of the active ingredients in such compositions.

U.S. Pat. No. 5,989,523 (Fitzjarrell) discloses a topical spray comprising niacinamide, Aloe Vera extract and NaPCA in a water carrier base. U.S. Pat. No. 5,910,312 (Fried) discloses an anti-acne composition comprising benzoyl peroxide, salicylic acid, and a vasoconstrictor in an inert carrier. Benzoyl peroxide has been suggested for treating acne vulgaris (See U.S. Pat. No. 4,387,107). For many years, benzoyl peroxide has been proven to be a particularly powerful keratolytic and anti-seborrhic agent, as well as being endowed with antibacterial properties. Topical benzoyl peroxide compositions, including a vehicle to enhance the efficacy thereof, are known (See U.S. Pat. No. 4,411,893). Topical compositions of benzoyl peroxide combination with antibiotics are also known. (See U.S. Pat. Nos. 4,407,794; 4,692,329 and 4,387,107). The problems of skin irritation from benzoyl peroxide or salicylic acid, and the chemical instability and reactivity of benzoyl peroxide are still not solved, although complex, dual-chamber delivery systems (such as U.S. Pat. No. 6,462,025; Vishnupad and U.S. Pat. No. 6,448,233; LaFevre et al.) have been disclosed. Such delivery systems are usually expensive, not convenient, and not precise in delivering product quantity.

Topical application of artemisinin for the treatment of viral tumors/diseases, hemorrhoids, and bullous skin diseases has been disclosed by Thornfeldt (U.S. Pat. No. 4,978,676).

Mazzio et al., (U.S. Patent Application Pub. No.: 2004185123) disclose a topical herbal formulation for preventing and/or treating dyshidrosis (pompholyx), non-responsive to topical steroids. The formulation may also be used to treat contact dermatitis, eczema, palmoplantar pustulosis and skin infections incurred by invasive pathogens such as mold, fungus and bacteria. The formulation is comprised of plant extracts and niacin, that when combined yield an effective multi-faceted pharmaceutical approach to treating dry skin disorders. The active ingredients within the formula include a combination of dry, aqueous, acid and alcohol extracts of black walnut hull (Ouglans Nigra), wormwood (Artemisia Absinthium), tumeric rhizome (Curcuma Longa), garlic (Allium sativum), chamomile (Matricaria Chamomile), licorice root (Glycyrrhiza Glabra), St. Johns wort (Hypericum perforatum), aloe vera, niacin and herbal anti-bacterial agents.

The present inventors have disclosed in US Patent Application Pub. No.: 20100120907 topical formulations comprising an Amyris alcohol and/or ester derivatives of Amyris alcohol which may be used for the treatment of diseases including herpes virus infection (e.g., HSV-1, HSV-2), epidermoid carcinoma, cold sores, and human papillomavirus. Amyris alcohols contemplated for use with the above invention includes valerianol, beta-eudesmol, epi-gamma-eudesmol, elemol, alpha-eudesmol, and ester derivatives thereof.

The present inventors have now discovered in a highly unexpected manner that amyris alcohol is efficient in the treatment of acne too. The present invention discloses compositions comprising amyris alcohol to treat acne in humans.

### SUMMARY OF THE INVENTION

The present invention provides novel use of amyris alcohol and esters of amyris alcohol to treat certain disease conditions in humans and other mammals.

In view of their high lipophilicity, non-irritation to the skin and stability, the esters of amyris alcohol would be desirable when administered topically. In addition, because of their stability and non-toxic nature, these agents can be made more readily available to the general pubic. The inventor has surprisingly and unexpectedly discovered that amyris alcohol and its esters have therapeutic utility in treating acne in humans.

The ester of amyris alcohol can be obtained by esterification of amyris alcohol by known synthetic procedures. In some embodiments, the ester is amyris acetate, which can be obtained by acetylation of amyris alcohol. In some embodiments, the ester of amyris alcohol is selected from the group consisting of amyris acetate, amyris propionate, amyris butanoate, amyris palmitate and amyris stearate.

It is believed that the prior art does not disclose or indicate that the ester of amyris alcohol has any utility as pro-drug forms suitable for oral and topical delivery for treating acne.

The present invention also generally pertains to pharmaceutical or cosmetic compositions comprising amyris alcohol or esters of amyris alcohol such as amyris acetate.

Accordingly, one aspect of the present invention relates to compositions comprising amyris alcohol or esters of amyris alcohol such as amyris acetate for use in the treatment of acne in humans.

In another aspect of this invention, the invention relates to esters of the active component or components of the amyris alcohol, namely eudesmol and valerianol for use in the treatment of acne in humans.

In particular, the amyris alcohol or esters of amyris alcohol such as amyris acetate described herein may be used for the preparation of therapeutic or cosmetic compositions in the treatment of acne in humans. In some embodiments, the compositions useful in the method may be topically applied to the human in need of such therapy.

In some embodiments, the methods of the present invention neither destroy healthy, uninfected tissue nor result in any local or systemic side effects, scarring, disfigurement or discomfort to the human that is being treated. The method comprises administering an effective amount of amyris alcohol or ester of amyris alcohol to an area of the human which has acne or is at risk of developing acne in order to eliminate the acne or prevent it from forming.

In accordance with some embodiments, regular use of the amyris alcohol or ester of amyris alcohol is meant to mean application of the alcohol or ester at least once a day to the body surface containing acne.

There is further disclosed a method for the prevention and treatment acne, comprising administering a cream or douche containing amyris alcohol or an ester of amyris alcohol or mixtures thereof, to the affected area of the human body.

In some embodiments, the pharmaceutical or cosmetic compositions of the present invention can further comprise phytoestrogens, antioxidants, anti-bacterial agents and anti-viral agents.

Accordingly, another aspect of the invention is to disclose the pharmaceutical or cosmetic compositions comprising amyris alcohol or ester of amyris alcohol such as amyris acetate, the phytoestrogens, namely, glabridin and miroestrol, the anti-oxidant, namely, tetrahydrocurcumin and the anti-bacterial agent, namely, artemisinin or dihydroartemisin.

There is further disclosed a method for the prevention and treatment acne, comprising administering a cream or douche comprising amyris alcohol or ester of amyris alcohol such as amyris acetate or mixtures thereof, glabridin, miroestrol, tetrahydrocurcumin and dihydroartemisinin, to the affected area of the human body.

The pharmaceutical or cosmetic compositions of the present invention can additionally include one or more pharmaceutically acceptable excipients. One of ordinary skill in the art would be familiar with pharmaceutically acceptable excipients. For example, the pharmaceutically acceptable excipient may be a water soluble sugar, such as mannitol, sorbitol, fructose, glucose, lactose, and sucrose.

The pharmaceutical or cosmetic compositions of the present invention may further comprise one or more pharmaceutically acceptable antioxidants. Any pharmaceutically acceptable antioxidant known to those of ordinary skill in the art is contemplated for inclusion in the present pharmaceutical or cosmetic compositions. For example, the pharmaceutically acceptable antioxidant may be selected from the group consisting of ascorbic acid, sodium ascorbate, sodium bisulfate, sodium metabisulfate and monothio glycerol.

The pharmaceutical or cosmetic compositions of the present invention may further comprise one or more pharmaceutically acceptable preservatives. Any pharmaceutically acceptable preservative known to those of ordinary skill in the art is contemplated for inclusion in the present pharmaceutical or cosmetic compositions. Examples of such preservatives include methylparaben, methylparaben sodium, propylparaben, propylparaben sodium, benzalkonium chloride, and benzthonium chloride.

The pharmaceutical or cosmetic compositions of the present invention may further comprise one or more pharmaceutically acceptable buffering agents. Any pharmaceutically acceptable buffering agent known to those of ordinary skill in the art is contemplated for inclusion in the present pharmaceutical or cosmetic compositions. Examples of such buffering agents include of monobasic sodium phosphate, dibasic sodium phosphate, sodium benzoate, potassium benzoate, sodium citrate, sodium acetate, and sodium tartrate.

The pharmaceutical or cosmetic compositions of the present invention can include any concentration of a compound of the present invention. For example, the concentration of compound may be 0.1% by weight to 20% by weight or greater. In certain particular embodiments, the concentration of amyris alcohol or amyris acetate is 5% to 15% by weight. Similarly, the composition may additionally contain between 2 μg/g and 20 μg/g of miroestrol, between 1% to 5% of tetrahydrocurcumin, between 1% and 5% of dihydroartemisinin and between 0.2% and 2% of glabridin.

In some embodiments of the present invention, the pharmaceutical or cosmetic composition includes more than one of the compounds set forth above. In other embodiments of the present invention, the pharmaceutical or cosmetic composition includes one or more secondary therapeutic agents directed to a disease or health-related condition, as discussed below.

The present invention also generally pertains to methods of treating or preventing acne in a subject, comprising providing a therapeutically effective amount of any of the pharmaceutical or cosmetic compositions set forth above, and administering the composition to the subject. The subject can be any subject, such as a mammal. In certain particular embodiments, the mammal is a human. The human may be an individual affected by or at risk of developing acne amenable to therapy with amyris alcohol.

The pharmaceutical or cosmetic composition of the present invention may be administered to the subject by any method known to those of ordinary skill in the art. For example, the method of administering the composition to the subject may include oral, topical, nasal, inhalational, rectal, or vaginal. Methods of administration are discussed in greater detail in the specification below.

In certain embodiments of the methods of the present invention, the method involves administering to the subject a therapeutically effective amount of a secondary agent. The secondary agent can be any pharmacologic agent known or suspected to be of benefit in the treatment or prevention of a disease or health-related condition in a subject. For example, in some embodiments, the secondary agent is a secondary antihyperproliferative agent. Secondary antihyperproliferative agents, which include chemotherapeutic agents, are well-known to those of ordinary skill in the art. Examples of such agents include doxorubucin, daunorubicin, mitomycin, actinomycin D, bleomycin, cisplatin, VP16, an enedyine, taxol, vincristine, vinblastine, carmustine, mellphalan, cyclophsophamide, chlorambucil, busulfan, lomustine, 5-fluorouracil, gemcitabin, BCNU, or camptothecin. The secondary agent may be an anti-viral agent. Examples of anti-viral agents include acyclovir, tetracaine, penciclovir, docosanol, and valacyclovir.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. The Chemical Structures of Eudesmol, Valerianol and Elemol.

FIG. 2. Chemical structure of Dihydroartemisinin.

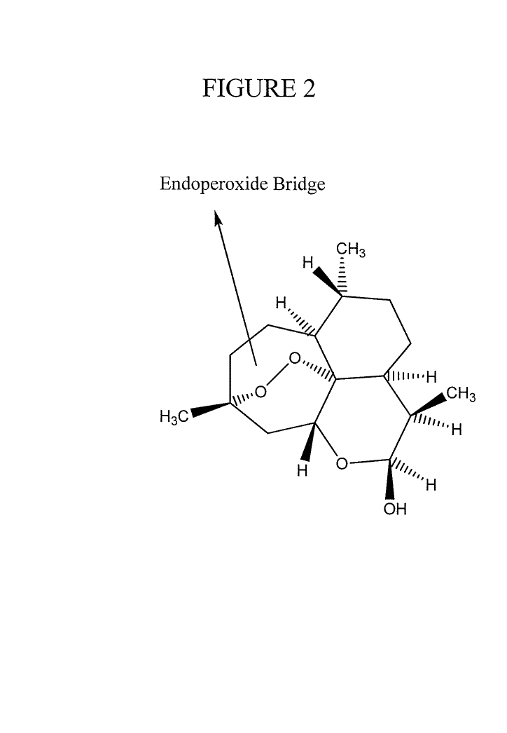

FIG. 3. The Chemical Structure of Glabridin.

FIG. 4. The chemical structure of miroestrol and deoxymiroestrol.

FIG. 5. The Chemical Structure of Tetrahydrocurcumin.

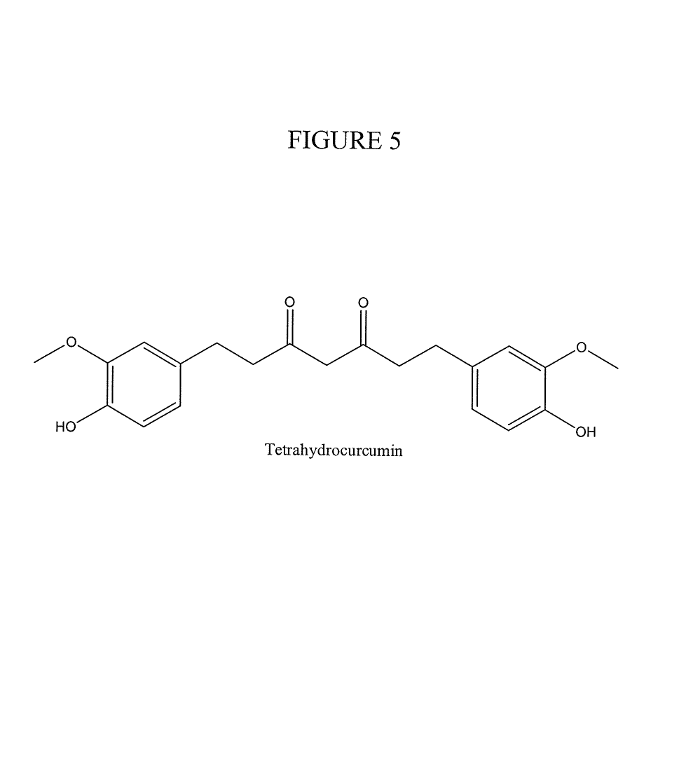

FIG. 6. Examples of esterified amyris alcohols.

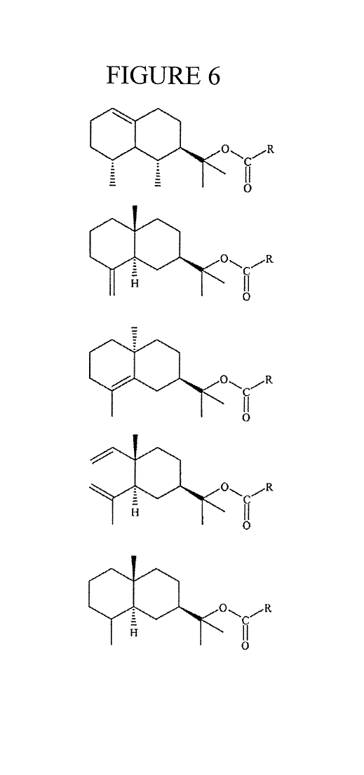

### DETAILED DESCRIPTION

The present invention is based on the inventors' highly unexpected discovery of the use of amyris alcohol or certain esters of amyris alcohol that are highly lipophilic, for treating acne in humans. These compounds are suitable by any route of administration, but are particularly topical administration in view of their lipid solubility. These compounds thus provide for a novel form of therapy for treating any acne. Further, the amyris alcohol or its ester is combined with phytoestrogens such as glabridin and miroestrol, antioxidants such as tetrahydrocurcumin and anti-bacterial agents such as the naturally occurring peroxides, namely, artemisinin and dihydroartemisinin for improved therapeutic effect in treating acne.

Before describing the present invention in detail, it is to be understood that this invention is not limited to particular drugs or drug delivery systems, as such may vary. It is also to be understood that the terminology used herein is for the purpose of describing particular embodiments only, and is not intended to be limiting.

It must be noted that, as used in this specification, the singular forms “a,” “an” and “the” include plural referents unless the context clearly dictates otherwise. Thus, for example, reference to “a pharmacologically active agent” includes a combination of two or more pharmacologically active agents, and the like. In describing the present invention, the following terminology will be used in accordance with the definitions set out below.

The terms “active agent,” “drug” and “pharmacologically active agent” are used interchangeably herein to refer to a chemical material or compound which, when administered to an organism (human or animal) induces a desired pharmacologic effect. Included are derivatives and analogs of those compounds or classes of compounds specifically mentioned which also induce the desired pharmacologic effect.

The phrases “pharmaceutical,” “pharmaceutically,” or “pharmacologically acceptable” refer to molecular entities and compositions that do not produce an unacceptably adverse, allergic or other untoward reaction when administered to an animal, or human, as appropriate. As used herein, “pharmaceutical” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredients, its use in the therapeutic compositions is contemplated. Supplementary active ingredients to treat the disease of interest, such as anti-inflammatory agents, can also be incorporated into the compositions.

The term “topical administration” is used in its conventional sense to mean delivery of a topical drug or pharmacologically active agent to the skin or mucosa.

The terms “carriers” or “vehicles” as used herein refer to carrier materials suitable for drug administration. Carriers and vehicles useful herein include any such materials known in the art, e.g., any liquid, gel, solvent, liquid diluent, solubilizer, or the like, which is nontoxic and which does not interact with other components of the composition in a deleterious manner.

By an “effective” amount of a drug or pharmacologically active agent is meant a nontoxic but sufficient amount of the drug or agent to provide the desired effect.

The term “amyris alcohol” as used herein refers to the alcohol distilled from the amyris oil by vacuum distillation. The volatile organic compounds are distilled off and the alcohols are concentrated due to their higher boiling point. Thus amyris alcohol is a mixture of alcohols present in amyris oil.

The term “ester of amyris alcohol” refers to the fully acylated or esterified product of amyris alcohol. The various alcohols present in the amyris alcohol are almost fully esterified and thus “ester of amyris alcohol” contains a mixture of esters.

The term “eudesmol” as used herein is intended to encompass not only α-, β- and γ-eudesmol, but any isomer or any compounded mixture thereof.

The term “acne” is used herein as a general term to include inflammatory diseases of the pilosebaceous unit. In the medical field, the specific type of acne is usually indicated by a modifying term, although the term acne is frequently used alone to designate common acne or acne vulgaris.

The term “cosmetic composition” as used herein refers to any substance or preparation intended to be placed in contact with the various external parts of the human body with a view exclusively or mainly to cleaning them, perfuming them, changing their appearance, and/or correcting body odours, and/or protecting them or keeping them in good condition. Such compositions must not be harmful to human health when they are applied under normal or foreseeable conditions of use.

The term “tetrahydrocurcumin” as used herein refers to not only tetrahydrocurcumin but all of tetrahydrocucuminoids which is a mixture of tetrahydrocurcumin, tetrahydrodemethoxycurcumin tetrahydrobisdemothoxycurcumin which are obtained from hydrogenation of tetrahydrocurcuminoids.

I. Description of Chemical Compounds Involved in the Present Invention

A. Amyris Alcohol and its Ester Derivatives

A member of the Rutaceae family, amyris tree (Amyris balsamifera) is native to Haiti but is now grown in tropical zones throughout the world. Amyris essential oil (Van T B A, Kleis R, et. al. (1989); Essential oil of Amyris balsamifera, Phytochemistry 28(7): 1909-1912) commonly referred to as West Indian Amyris alcohol, the Botanical origin of the tree yielding this oil remained obscure until 1886. The main country of origin today is Haiti, where the oil is obtained by steam distillation from broken up wood & branches and its chemical composition has been investigated (Van T B A et al. (1991): Valerianol—The major sesquiterpene alcohol from Amyris balsamifera, J. Essent. Oil Res., 3, 59-60; Van T B A et al. (1991), 7-Epi-α-eudesmol: A rare sesquiterpene alcohol, J. Essent. Oil Res., 3, 127-128). Amyris essential oil has been used for wound washes, influenza, child birth recovery, diarrhea, used also as a room fragrance or mood fragrance, as a cheaper alternative to genuine sandalwood oil. It is used as a fragrance, fixative or a component of soap fragrance.

Amyris oil is rich in sesquiterpene alcohols (60-80%), e.g. valerianol, eudesmol (α, β and γ isomers) and elemol (Table 1; Bauer K. Garbe D, Surburg H. Common fragrance and flavor materials: preparation, properties and uses. 2nd ed. Weinheim: VCH; 1990). The volatile compounds had been identified in the leaf oil of Amyris balsamifera from Cuba (Pino, J A, et al. (2006): Aromatic Plants from Western Cuba. VI. Composition of the Leaf Oils of Murraya exotica L, Amyris balsamifera L., Severinia buxifolia (Poir.) Ten. and Triphasia trifolia (Burm. f), Journal of Essential Oil Research: JEOR, January/February 2006). Fifty-six constituents were identified which constituted more than 95% of the oil composition. The oil was dominated by sesquiterpene alcohols, particularly by valerianol (43.8%), with lesser amounts of γ-eudesmol (15.4%).

TABLE 1
Typical gas chromatography analysis of Amyris Oil
(Alcohol Content from 2 different manufacturers)

| Constituent | Percentage | Percentage |
|---|---|---|
| elemol | 8.99 | 10.0 |
| eudesmol | 42.12 | 27.7 |
| valerianol | 17.04 | 22.1 |
| Selin-5-en-7-ol | | 6.4 |
| Total Alcohol Content | 69.45 | 66.2 |

(Source: Green Valley Aromatherapy Ltd., 420 Fitzgerald Avenue, Courtenay, British Columbia, CANADA V9N7N2)

β-eudesmol, is known to have various unique effects on the nervous system. β-eudesmol at concentrations of 100 and 150 μM significantly induced neurite extension in PC-12 cells, which was accompanied, at the highest concentration, by suppression of [³H]thymidine incorporation. Beta-Eudesmol, being a small molecule, may therefore be a promising lead compound for potentiating neuronal function (Yutaro O, et al., (2002); Beta-Eudesmol Induces Neurite Outgrowth in Rat Pheochromocytoma Cells Accompanied by an Activation of Mitogen-Activated Protein Kinase, Pharmacology and Experimental Therapeutics, 301: 803-811). Proliferation of porcine brain microvascular endothelial cells and human umbilical vein endothelial cells (HUVEC) was inhibited by β-eudesmol (50-100 microM). It also inhibited the HUVEC migration stimulated by basic fibroblast growth factor (bFGF) and the tube formation by HUVEC in Matrigel. β-eudesmol (100 microM) blocked the phosphorylation of extracellular signal-regulated kinase (ERK) 1/2 induced by bFGF or vascular endothelial growth factor. Furthermore, β-eudesmol significantly inhibited angiogenesis in subcutaneously implanted Matrigel plugs in mice and in adjuvant-induced granuloma in mice. These results indicate that β-eudesmol inhibits angiogenesis, at least in part, through the blockade of the ERK signaling pathway (Tsuneki H., et al., Antiangiogenic activity of beta-eudesmol in vitro and in vivo, Eur J Pharmacol. 2005; 512(2-3):105-15). It has been shown that β-Eudesmol blocks the neuromuscular junction. Like phencyclidine, β-eudesmol blocked the nicotinic ACh receptor channel in both the open and closed conformations, and accelerated the desensitization of the nicotinic ACh receptor (Kimura M, et al., (1991); Mechanism of the blocking action of β-eudesmol on the nicotinic acetylcholine receptor channel in mouse skeletal muscles. Neuropharmacology 30: 835-841).

α-eudesmol potently inhibits the presynaptic omega-agatoxin IVA-sensitive (P/Q-type) Ca²⁺ channel and neurogenic inflammation following electrical stimulation of rat trigeminal ganglion. It has been suggested that the omega-agatoxin IVA-sensitive Ca²⁺ channel blocker, α-eudesmol, may become useful for the treatment of the neurogenic inflammation in the trigemino-vascular system such as migraine (Asakura K., et al., (2000); x-Eudesmol, a P/Q-type Ca²⁺ channel blocker, inhibits neurogenic vasodilation and extravasation following electrical stimulation of trigeminal ganglion. Brain Res 873: 94-101). α-eudesmol is a Ca²⁺ channel blocker and neurogenic vasodilator, which is useful for treatment of neurogenic inflammation in trigemino-vascular system such as migraine (Asakura K., et al., (2000); Omega-agatoxin IVA-sensitive Ca(2+) channel blocker, alpha-eudesmol, protects against brain injury after focal ischemia in rats. Eur. J. Pharmacol. 7, 57-65). It attenuates post-ischemic brain injury by reducing the extra cellular glutamate. β-eudesmol is an antidote for intoxication from organophosphorous anti-choline esterase agents (Chiou L. C., et al., (1995); Beta-eudesmol as an antidote for intoxication from organo-phosphorus anticholine esterase agents. Eur. J. Phar-macol. 13, 151-156). It could be used as anti-epileptic (Chiou L C., et al., (1997); Chinese herb constituent beta-eudesmol alleviated the electro-shock seizures in mice and electrographic seizures in rat hippocampal slices. Neurosci. Lett. 15, 171-174), antiangiogenic activity (Tsuneki H., et al., (2005); Antiangiogenic activity of beta-eudesmol in vitro and in vivo, Eur J Pharmacol. 512(2-3):105-15). β-Eudesmol inhibited Na⁺, K(+)-ATPase activity most strongly among the various kinds of phosphatases examined (Satoh K, et al., (1992); Inhibition of Na⁺, K⁺-ATPase activity by beta-eudesmol, a major component of Atractylodis lanceae rhizoma, due to the interaction with enzyme in the Na, E1 state. Biochem Pharmacol 44: 373-378).

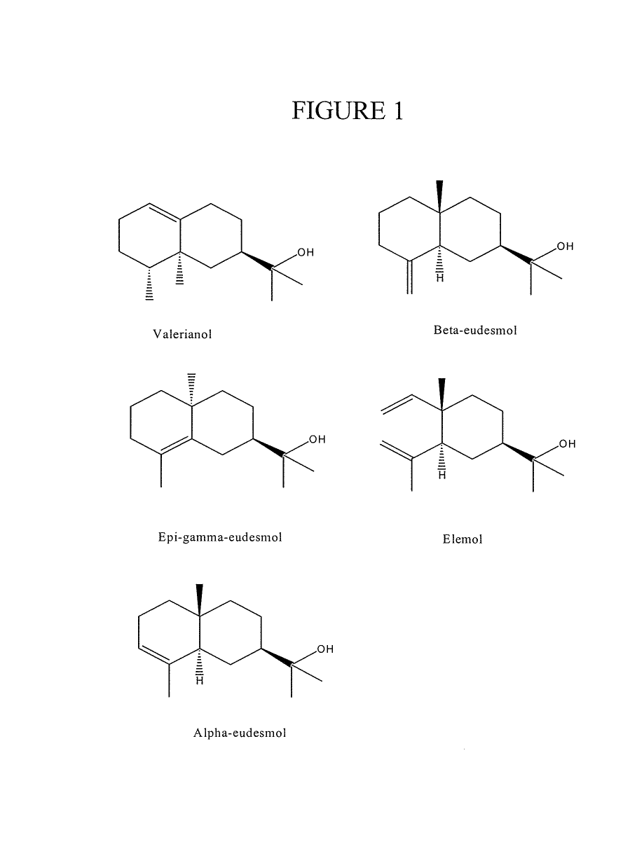

The chemical formula for eudesmol is C₁₅H₂₆O and that of valerianol is C₁₅H₂₆O and the chemical structures are shown in FIG. 1.

The alcohols present in amyris oil, hereinafter will be called as amyris alcohol, may be obtained by fractional distillation of the oil under vacuum by removing the volatile terpenes which are low boiling than the alcohols present in the oil, with the eudesmol and valerianol appearing in different ratios. The amyris alcohol is colorless to pale yellow in appearance.

Amyris oil is commonly used in the flavor and fragrance industries as a replacement for sandalwood oil and is considered woody, cedar-like, warm and herbaceous. As such, they are non-toxic and harmless when used either for external application on the skin or internal consumption for flavor.

Since amyris alcohol contains 60-95% of alcohol, excess application to the skin may cause irritation and itching. To eliminate the irritation and itching, the alcohols can be esterified as they are milder to the skin. The preferred ester for the present invention is amyris acetate which is obtained by the acetylation of the amyris alcohol by known synthetic procedure.

The ester derivatives of amyris alcohol are capable of reverting to the active parent compound following enzymatic or chemical hydrolysis. These derivatives have a higher lipophilicity, lipid solubility and less irritation to the skin than the parent compound, and hence are better able to be incorporated into certain pharmaceutical or cosmetic formulations, including cream and ointment pharmaceutical or cosmetic formulations.

Various amyris alcohol derivatives, such as esterified amyris alcohols, may be used with the present invention. The ester derivatives of amyris alcohol are capable of reverting to the active parent compound following enzymatic or chemical hydrolysis. These derivatives may have a higher lipophilicity, lipid solubility and be less irritating to the skin than the corresponding amyris alcohol or parent compound. Hence, these ester derivatives may be better suited for incorporation into certain pharmaceutical formulations, such as cream and ointment pharmaceutical formulations. The compounds of the present invention are set forth by the following formulae:

R—CO—O₁Am (I)

wherein O₁Am refers to an oxygen present in an alcohol group of the corresponding unesterified amyris alcohol.

In formula I, R may be an alkyl group, an aryl group, an alkylene group (e.g., an alkenyl or alkynyl), aralalkyl, heteroalkyl, or an arylene group, each of which may vary in size, e.g., C₁-C₂₂, C₁-C₁₈, C₁-C₁₂, C₁-C₆. In certain embodiments, the aryl or aralkyl group is C≦22, C≦18, C≦12, C=6 or C=7. The alkyl, aryl and alkylene groups may be substituted or unsubstituted, branched or straight chains. In addition, R may contain heteroatoms and may be straight chained or branched.

Examples of suitable straight-chain alkyl groups in formula I include methyl, ethyl, propyl, butyl, hexyl, heptyl, octyl, dodecyl, 1-pentadecyl, 1-heptadecyl and the like groups. Examples of suitable branched chain alkyl groups in formula I include isopropyl, sec-butyl, t-butyl, 2-methylbutyl, 2-pentyl, 3-pentyl and the like groups. Examples of suitable cyclic alkyl groups in formula I include cyclopropyl, cyclobutyl, cyclopentyl and cyclohexyl groups.

Examples of suitable “alkenyl” groups in formula I include vinyl(ethenyl), 1-propenyl, i-butenyl, pentenyl, hexenyl, n-decenyl and c-pentenyl and the like.

The groups may be substituted, generally with 1 or 2 substituents, wherein the substituents are independently selected from halo, hydroxy, alkoxy, amino, mono- and dialkylamino, nitro, carboxyl, alkoxycarbonyl, and cyano groups.

By the expression “phenalkyl groups wherein the alkyl moiety contains 1 to 3 or more carbon atoms” is meant benzyl, phenethyl and phenylpropyl groups wherein the phenyl moiety may be substituted. When substituted, the phenyl moiety of the phenalkyl group may contain independently from 1 to 3 or more alkyl, hydroxy, alkoxy, halo, amino, mono- and dialkylamino, nitro, carboxyl, alkoxycarbonyl and cyano groups.

Examples of suitable “heteroaryl” in formula I are pyridinyl, thienyl or imidazolyl.

As noted herein, the expression “halo” is meant in the conventional sense to include F, Cl, Br, and I.

Among the compounds represented by the general formula I, preferred compounds are such in which R is one of the following groups: methyl, ethyl, propyl, butyl, pentyl, hexyl, 1-pentadecyl, 1-heptadecyl, isobutyl, methoxyethyl, ethoxyethyl, benzyl and nicotinyl.

Without wishing to be bound by any theory, it is anticipated that the esterified amyris alcohols may be enzymatically cleaved once administered to a mammal or human patient in vivo.

### Methods of Synthesis

The compounds can be prepared by any method known to those of ordinary skill in the art. For example, the compounds of the present invention are esters of alcohols which are the constituents of amyris alcohol. Various methods have been described in the literature pertaining to the synthesis of a number of esters of carboxylic acids and alcohols (e.g., March's Advanced Organic Chemistry: Reactions, Mechanisms, and Structure, 5th Edition, by Michael B. Smith and Jerry March, John. Wiley and Sons, Inc, 2001, which is incorporated by reference in its entirety). Amyris alcohols and/or esterified amyris alcohols may be purified and used with the present invention.

Various approaches may be used to produce esterified amyris alcohols. Since amyris alcohol is a mixture of tertiary alcohols, esterification can be accomplished using the following procedure. The alcohol can be converted to lithium alcoholate using equimolar amount of either methyl-lithium or t-butyl-lithium under dry and nitrogen atmosphere. The resulting alcoholate can be allowed to react with an equimolar amount of an acyl chloride in diethyl ether under dry condition to produce the desired ester quantitatively. The resulting ester can be vacuum distilled for further purification.

For example, the following protocol may be used in certain embodiments to produce esterified amyris alcohols. A mixture of 100 ml (.about.0.4M of alcohol content) of amyris alcohol (Texarome Inc, Leakey, Tex.), 190 ml (2M) of acetic anhydride and 5 drops of H₃PO₄ (85% in water) may be introduced in a 1000 ml flask, and the mixture may be stirred over night, at room temperature. Afterwards, 2 L of water may be added and the stirring may be prolonged for an additional period of 2 hours. The crude product may be extracted by washing the water solution with 1 L of n-hexane. The organic phase thus obtained may be washed twice with a saturated NaHCO₃ water solution, then twice with brine and finally dried over anhydrous MgSO₄ and concentrated. 130 g of crude product (95% yield) having a GC purity of >90% may be obtained using this approach. In various embodiments, propionic anhydride may be used instead of acetic anhydride.

### Chemical Group Definitions

As used herein, “hydrogen” means —H; “hydroxy” means —OH; “oxo” means ═O; “halo” means independently —F, —Cl, —Br or —I; “amino” means ═NH.sub.2 (see below for definitions of groups containing the term amino, e.g., alkylamino); “hydroxyamino” means —NHOH; “nitro” means —NO₂; imino means ═NH (see below for definitions of groups containing the term imino, e.g., alkylamino); “cyano” means —CN; “azido” means —N₃; “mercapto” means —SH; “thio” means ═S; “sulfonamido” means —NHS(O)₂— (see below for definitions of groups containing the term sulfonamido, e.g., alkylsulfonamido); “sulphonyl” means —S(O)₂— (see below for definitions of groups containing the term sulphonyl, e.g., alkylsulphonyl); and “silyl” means —SiH³ (see below for definitions of group(s) containing the term silyl, e.g., alkylsilyl).

For the groups below, the following parenthetical subscripts further define the groups as follows: “(Cn)” defines the exact number (n) of carbon atoms in the group; “(C≦n)” defines the maximum number (n) of carbon atoms that can be in the group; (Cn-n′) defines both the minimum (n) and maximum number (n′) of carbon atoms in the group. For example, “alkoxy_{(C≦10)}” designates those alkoxy groups having from 1 to 10 carbon atoms (e.g., 1, 2, 3, 4, 5, 6, 7, 8, 9, or 10, or any range derivable therein (e.g., 3-10 carbon atoms)). Similarly, “alkyl_{(C2-10)}” designates those alkyl groups having from 2 to 10 carbon atoms (e.g., 2, 3, 4, 5, 6, 7, 8, 9, or 10, or any range derivable therein (e.g., 3-10 carbon atoms)).

The term “alkyl” when used without the “substituted” modifier refers to a non-aromatic monovalent group, having a saturated carbon atom as the point of attachment, a linear or branched, cyclo, cyclic or acyclic structure, no carbon-carbon double or triple bonds, and no atoms other than carbon and hydrogen. The groups, —CH₃ (Me), —CH₂CH₃ (Et), —CH₂CH₂CH₃ (n-Pr), —CH(CH₃)₂ (iso-Pr), —(CH₂)₂ (cyclopropyl), —CH₂CH₂CH₂CH₃ (n-Bu), —CH(CH₃)CH₂CH₂ (sec-butyl), CH₂CH(CH₃)2 (iso-butyl), —C(CH₃)₃ (tert-butyl), —CH₂C(CH₃)₃ (neo-pentyl), cyclobutyl, cyclopentyl, cyclohexyl, and cyclohexylmethyl are non-limiting examples of alkyl groups. The term “substituted alkyl” refers to a non-aromatic monovalent group, having a saturated carbon atom as the point of attachment, a linear or branched, cyclo, cyclic or acyclic structure, no carbon-carbon double or triple bonds, and at least one atom independently selected from the group consisting of N, O, F, Cl, Br, I, Si, P, and S. The following groups are non-limiting examples of substituted alkyl groups: —CH₂OH, —CH₂Cl, —CH₂Br, —CH₂SH, —CF₃, —CH₂CN, —CH₂C(O)H, —CH₂C(O)OH, —CH₂C(O)OCH₃, —CH₂C(O)NH₂, —CH₂C(O)NHCH₃, —CH₂2C(O)CH₃, —CH₂OCH₃, —CH₂OCH₂CF₃, —CH₂OC(O)CH₃, —CH₂NH₂, —CH₂NHCH₃, —CH₂N(CH₃)₂, —CH₂CH₂Cl, —CH₂CH₂OH, —CH₂CF₃, —CH₂CH₂OC(O)CH₃, —CH₂CH₂NHCO₂C(CH₃)₂, and —CH₂Si(CH₃)₃.

The term “alkenyl” when used without the “substituted” modifier refers to a monovalent group, having a nonaromatic carbon atom as the point of attachment, a linear or branched, cyclo, cyclic or acyclic structure, at least one nonaromatic carbon-carbon double bond, no carbon-carbon triple bonds, and no atoms other than carbon and hydrogen. Non-limiting examples of alkenyl groups include: —CH═CH₂ (vinyl), —CH═CHCH₃, —CH═CHCH₂CH₃, —CH₂CH═CH₂ (allyl), —CH₂CH═CHCH₃, and —CH═CH—C₆H₅. The term “substituted alkenyl” refers to a monovalent group, having a nonaromatic carbon atom as the point of attachment, at least one nonaromatic carbon-carbon double bond, no carbon-carbon triple bonds, a linear or branched, cyclo, cyclic or acyclic structure, and at least one atom independently selected from the group consisting of N, O, F, Cl, Br, I, Si, P, and S. The groups, —CH═CHF, —CH═CHCl and —CH═CHBr, are non-limiting examples of substituted alkenyl groups.

The term “alkynyl” when used without the “substituted” modifier refers to a monovalent group, having a nonaromatic carbon atom as the point of attachment, a linear or branched, cyclo, cyclic or acyclic structure, at least one carbon-carbon triple bond, and no atoms other than carbon and hydrogen. The groups, —C≡CH, —C≡CCH₃, —C≡CC₆H₅ and —CH₂C≡CCH₃, are non-limiting examples of alkynyl groups. The term “substituted alkynyl” refers to a monovalent group, having a nonaromatic carbon atom as the point of attachment and at least one carbon-carbon triple bond, a linear or branched, cyclo, cyclic or acyclic structure, and at least one atom independently selected from the group consisting of N, O, F, Cl, Br, I, Si, P, and S. The group, —C≡CSi(CH₃)₃, is a non-limiting example of a substituted alkynyl group.

The term “aryl” when used without the “substituted” modifier refers to a monovalent group, having a aromatic carbon atom as the point of attachment, said carbon atom forming part of a six-membered aromatic ring structure wherein the ring atoms are all carbon, and wherein the monovalent group consists of no atoms other than carbon and hydrogen. Non-limiting examples of aryl groups include phenyl (Ph), methylphenyl, (dimethyl)phenyl, —C₆H₄CH₂CH₃ (ethylphenyl), —C₆H₄CH₂CH₂CH₃ (Propylphenyl), —C₆H₄CH(CH₃)₂, —C₆H₄CH(CH₂)₂, —C₆H₃(CH₃)CH₂CH₃ (methylethylphenyl), —C₂H₄CH═CH₂ (vinylphenyl), —C₆H₄CH═CHCH₃, —C₆H₄C≡CH, —C₆H₄C≡CCH₃, naphthyl, and the monovalent group derived from biphenyl. The term “substituted aryl” refers to a monovalent group, having a aromatic carbon atom as the point of attachment, said carbon atom forming part of a six-membered aromatic ring structure wherein the ring atoms are all carbon, and wherein the monovalent group further has at least one atom independently selected from the group consisting of N, O, F, Cl, Br, I, Si, P, and S, Non-limiting examples of substituted aryl groups include the groups: —C₆H₄F, —C₆H₄Cl, —C₆H₄Br, —C₆H₄I, —C₆H₄OH, —C₆H₄OCH₃, —C₆H₄OCH₂CH₃, —C₆H₄OC(O)CH₃, —C₆H₄NH₂, —C₆H₄NHCH₃, —C₆H₄N(CH₃)₂, —C₆H₄CH₂OH, —C₆H₄CH₂OC(O)CH₃, —C₆H₄CH₂NH₂, —C₆H₄CF₃, —C₆H₄CN, —C₆H₄CHO, —C₆H₄CHO, —C₆H₄C(O)CH₃, —C₆H₄C(O)C₆H₅, —C₆H₄CO₂H, —C₆H₄CO₂CH₃, —C₆H₄CONH₂, —C₆H₄CONHCH₃, and —C₆H₄CON(CH₃)₂.

The term “aralkyl” when used without the “substituted” modifier refers to the monovalent group—alkanediyl-aryl, in which the terms alkanediyl and aryl are each used in a manner consistent with the definitions provided above. Non-limiting examples of aralkyls are: phenylmethyl (benzyl, Bn), 1-phenyl-ethyl, 2-phenyl-ethyl, indenyl and 2,3-dihydro-indenyl, provided that indenyl and 2,3-dihydro-indenyl are only examples of aralkyl in so far as the point of attachment in each case is one of the saturated carbon atoms. When the term “aralkyl” is used with the “substituted” modifier, either one or both the alkanediyl and the aryl is substituted. Non-limiting examples of substituted aralkyls are: (3-chlorophenyl)-methyl, 2-oxo-2-phenyl-ethyl (phenylcarbonylmethyl), 2-chloro-2-phenyl-ethyl, chromanyl where the point of attachment is one of the saturated carbon atoms, and tetrahydroquinolinyl where the point of attachment is one of the saturated atoms.

The term “heteroaryl” when used without the “substituted” modifier refers to a monovalent group, having a aromatic carbon atom or nitrogen atom as the point of attachment, said carbon atom or nitrogen atom forming part of an aromatic ring structure wherein at least one of the ring atoms is nitrogen, oxygen or sulfur, and wherein the monovalent group consists of no atoms other than carbon, hydrogen, aromatic nitrogen, aromatic oxygen and aromatic sulfur. Non-limiting examples of aryl groups include acridinyl, furanyl, imidazoimidazolyl, imidazopyrazolyl, imidazopyridinyl, imidazopyrimidinyl, indolyl, indazolinyl, methylpyridyl, oxazolyl, phenylimidazolyl, pyridyl, pyrrolyl, pyrimidyl, pyrazinyl, quinolyl, quinazolyl, quinoxalinyl, tetrahydroquinolinyl, thienyl, triazinyl, pyrrolopyridinyl, pyrrolopyrimidinyl, pyrrolopyrazinyl, pyrrolotriazinyl, pyrroloimidazolyl, chromenyl (where the point of attachment is one of the aromatic atoms), and chromanyl (where the point of attachment is one of the aromatic atoms). The term “substituted heteroaryl” refers to a monovalent group, having a aromatic carbon atom or nitrogen atom as the point of attachment, said carbon atom or nitrogen atom forming part of an aromatic ring structure wherein at least one of the ring atoms is nitrogen, oxygen or sulfur, and wherein the monovalent group further has at least one atom independently selected from the group consisting of non-aromatic nitrogen, non-aromatic oxygen, non aromatic sulfur F, Cl, Br, I, Si, and P.

An “isomer” of a first compound is a separate compound in which each molecule contains the same constituent atoms as the first compound, but where the configuration of those atoms in three dimensions differs.

“Pharmaceutically acceptable salts” means salts of compounds of the present invention which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylicacids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, Selection and Use (P. H. Stahl & C. G. Wermuth eds., Verlag Helvetica Chimica Acta, 2002), which is incorporated herein by reference.

“Prevention” or “preventing” when used in reference to a disease includes: (1) inhibiting the onset of the disease in a subject or patient which may be predisposed to the disease but does not yet experience or display the pathology or symptomatology of the disease, (2) slowing the onset of the pathology or symptomatology of the disease in a subject of patient which may be predisposed to the disease but does not yet experience or display the pathology or symptomatology of the disease.

“Prodrug” means a compound that is convertible in vivo metabolically into an inhibitor according to the present invention. For example, prodrugs of amyris alcohols are presented herein, and it is envisioned that a variety of amyris alcohol derivatives or prodrug may be used with the present invention. The prodrug itself may or may not also have activity with respect to a given target protein or therapeutic effect. For example, a compound comprising a hydroxy group may be administered as an ester that is converted by hydrolysis in vivo to the hydroxy compound. As described herein, amyris alcohol prodrugs such as esterified amyris alcohols are provided for the treatment of diseases including acne. Suitable esters that may be converted in vivo into hydroxy compounds include acetates, citrates, lactates, phosphates, tartrates, malonates, oxalates, salicylates, propionates, succinates, fumarates, maleates, methylene-bis-b-hydroxynaphthoates, gentisates, isethionates, di-p-toluoyltartrates, methanesulfonates, ethanesulfonates, benzenesulfonates, p-toluenesulfonates, cyclohexylsulfamates, quinates, esters of amino acids, and the like. Amyris alcohols may be esterified using any of these approaches, and it is envisioned that these esterified amyris alcohols may be used with the present invention (e.g., to treat a herpesvirus infection, etc.) Similarly, a compound comprising an amine group may be administered as an amide that is converted by hydrolysis in vivo to the amine compound.

The term “saturated” when referring to a atom means that the atom is connected to other atoms only by means of single bonds.

The terms “subject” and “patient” includes humans, primates and other mammals.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers.

“Treatment” or “treating” includes: (1) inhibiting a disease in an subject or patient that is experiencing or displaying the pathology or symptomatology of the disease (i.e., arresting further development of the pathology and/or symptomatology), and (2) ameliorating the disease in a subject or patient that is experiencing or displaying the pathology or symptomatology of the diseased (i.e., reversing the pathology and/or symptomatology). It is believed that the literature does not disclose or indicate that amyris alcohol and the esters of amyris alcohol or eudesmol or valerianol have any utility as pro-drug forms suitable for oral and topical delivery for treating acne.

In some embodiments, the ester of amyris alcohol is selected from the group consisting of amyris acetate, amyris propionate, amyris butanoate, amyris palmitate and amyris stearate.

B. Dihydroartemisinin

Artemisinin is extracted from a traditional Chinese medicinal plant, Artemesia annua or sweet wormwood, and can then be chemically converted into several derivatives. In China, where A. annua was first described, it is known as qinghao, and the active ingredient artemisinin is termed qinghaosu. Chinese healers have used A. annua for the treatment of malaria and other maladies since at least 2400 years ago. In 1967 Mao Tse-Tung's government ordered a systematic examination of the traditional Chinese herbal pharmacopoeia that led to the isolation of the compound qinghaosu, or Artemisinin in 1972. Artemisinin is currently extracted from dried leaves and inflorescences from A. annua, an annual herb that grows wild throughout China and Southeast Asia (Dhingra V, Rao K V, Narasu N L: Current status of artemisinin and its derivatives as antimalarial drugs. Life Sci 66:279-300, 2000).

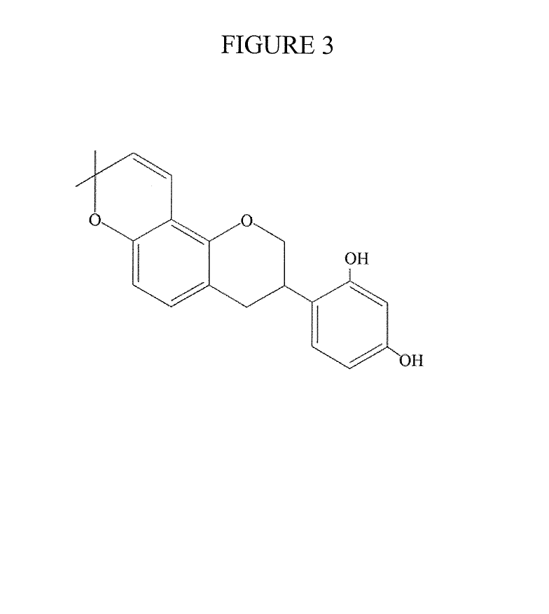

Dihydroartemisinin is the active metabolite of Artemisinin and is a sesquiterpene lactone (FIG. 3). The artemisinin molecule contains two oxygen atoms linked together in what is known as an ‘endoperoxide bridge’, which could react with an iron atom to form free radicals. Artemisinin is toxic to malaria parasites because the parasite contains a high amount iron in the form of heme molecules (Zhang F., Gosser D K Jr. et al., Hemin-catalyzed decomposition of artemisinin (qinghao), Biochem Pharmacol 1992; 43: 1805-9). Rapid growth abnormal cells sequester relatively large amount of iron mainly in the form of holotranferrin. Artemisinin has been shown to cause rapid and extensive damage to these abnormal cells and have reatively low toxicity to normal cells.

Artemisininin is virtually non-toxic (LD50=4228 mg/kg orally administered to mice) and without carcinogenicity. When artemisinin was tested with monkeys, they showed no toxicity after they received up to 292 mg/kg of artemether over 1 to 3 months. Very high doses of artemisinin when administered orally can produce neurotoxicity such as gait disturbances, loss of spinal and pain response, respiratory depression, and ultimately cardiopulmonary arrest in large animals (Smith S L., et al., The role of iron in neurotoxicity: a study of novel antimalarial drugs. Neurotoxicology 19:557-559, 1998). A 250-mg artemisinin was used in a pharmacokinetic study in healthy volunteers and was well-tolerated with no undesirable side effects (Benakis et al., Pharmacokinetics of artemisinin and artesunate after oral administration in healthy volunteers. American Journal of Tropical Medicine Hyg, January; 56(1):17-23, 1997).

C. Glabridin

Licorice (Glycyrrhiza glabra L.) is one of the most popular and widely consumed herbs in the world. In Ayurveda, it is used to relieve inflammations, eye diseases, throat infections, peptic ulcers, arthritic conditions, and liver diseases. Its effectiveness in treating these conditions is attributed to the expectorant, emollient, anti-inflammatory, antiviral, antihepatotoxic and antibacterial properties of its potent constituents. Licorice has been extensively researched for its medicinal and food uses because of its useful properties. The roots and stolons of the plant contain approximately 5-9% by weight glycyrrhizin, also known as glycyrrhizic or glycyrrhizinic acid. Glycyrrhizin is more than 50 times sweeter than cane sugar. Thus, it can be used as a natural sweetener.

Licorice has been used medicinally for its demulcent, diuretic, emollient, expectorant, laxative and estrogenic properties. Traditionally, licorice has also been used to relieve asthma, bronchitis, fevers ulcers, and cancers. Today, it is widely used to treat coughs and colds and as a digestive aid.

Licorice contains glycyrrhizin, saponins, asparagine, sugars, resin, bitter principles, a volatile oil, and other compounds. The main constituents of licorice are the triterpenoid saponin glycyrrhizin and a mixture of calcium and potassium salts of glycyrrhizinic acid. Other constituents include triterpenoid saponins (glabridin, glycyrrhetol, glabrolide, and isoglabrolide), isoflavones (formononetin, neoliquiritin, glabrone and hispaglabridin), triterpene sterols (onocerin, β-amyrin and stigmasterol), and coumarins (herniarin and umbelliferone). The hydrophobic fraction of licorice contains glabridin, the main ingredient, and several flavonoids, while the main constituents of the hydrophilic fraction are glycyrrhizin and glycyrrhetinic acid.

Glabridin (FIG. 3) is the main compound in the hydrophobic fraction of licorice extract. In a comprehensive study (Yokota T., et al., The inhibitory effect of glabridin from licorice extracts on melanogenesis and inflammation. Pigment Cell Res. 1998; 11(6):355-61), the inhibitory effects of glabridin on melanogenesis as well as inflammation were examined. The structure-function relationship of glabridin was also studied. Topical skin-depigmentation activities of the active component, glabridin, were examined using UVB-induced pigmented skins of brownish guinea pigs. A 0.5% glabridin solution was applied topically to the skin. Topical application of glabridin significantly reduced pigmentation induced by UVB radiation on the backs of the brownish guinea pigs. Skin samples were also taken from each of the glabridin treated areas for histological studies. The treated tissue was stained with 0.1% DOPA and the inhibition of melanogenesis was evaluated by counting the number of DOPA-positive melanocytes/mm² under an optical microscope. Epidermal histological studies performed showed that DOPA-positive melanocytes reduced in number on the skin treated with glabridin. Treatment with glabridin also lightened the skin color due to inhibition of melanogenesis. The authors concluded that the glabridin present in Licorice roots inhibits both melanin synthesis and inflammation. They also observed that these properties of glabridin were related to its structure.

Glabridin may inhibit melanogenesis by one of two mechanisms:
1. Inhibition of the production of active oxygen species: (O₂)
2. Inhibition of tyrosine: Human tyrosinase is an essential enzyme, which regulates the production of melanin, a group of brown to black pigments in the skin and eyes of humans.

It is a known fact that a number of reactions (e.g. inflammatory, etc.) are induced when human skin is exposed to UV radiation. The membrane phospholipids of the skin tissue are damaged by UV-induced active oxygen. Histological changes occur in the skin that manifest as erythemas and skin pigmentation. Active oxygen is one of the species that induces skin pigmentation. Thus, prevention of its production is linked to inhibition of melanogenesis. To test this, an assay was performed to study the inhibitory effect of glabridin on superoxide anion production. Glabridin inhibited superoxide (active oxygen) formation at concentrations from 0.33 μg/ml to 33.3 μg/ml (Fukai T., et al., Preliminary evaluation of antinephritis and radical scavenging activities of glabridin from Glycyrrhiza glabra. Fitoterapia. 2003; 74(7-8):624-9). Thus, glabridin may be useful for treating conditions like melasma or pigmentation of skin due to sun-exposure.

An assay was performed to test the anti-inflammatory activity of glabridin when used for topical application. UVB-induced pigmented skins of guinea pigs were treated with 0.5% glabridin solution. It was observed that glabridin decreased the inflammation induced by UVB irradiation on the skin. An assay was performed to determine the inhibitory effect of glabridin on cyclooxygenase activity. Cyclooxygenase is an enzyme that metabolizes arachidonic acid into prostaglandins, which are mediators that initiate the inflammatory cascade reaction. It was observed that an addition of 6.25 μg/ml glabridin inhibited the cyclooxygenase activity with respect to the control. The positive control in this experiment was indomethacin, a known cyclooxygenase inhibitor. It is believed that glabridin has the anti-inflammatory effect through the arachidonic acid cascade by inhibition to cyclooxygenase.

The estrogenic properties of glabridin were tested in view of the resemblance of its structure and lipophilicity to those of estradiol (Somjen D., et al., Estrogen-like activity of licorice root constituents: glabridin and glabrene, in vascular tissues in vitro and in vivo. J Steroid Biochem Mol Biol. 2004; 91(3):147-55). The results indicate that glabridin is a phytoestrogen, binding to the human estrogen receptor and stimulating creatine kinase activity in rat uterus, epiphyseal cartilage, diaphyseal bone, aorta, and left ventricle of the heart. The stimulatory effects of 2.5-25 μg/animal glabridin were similar to those of 5 μg/animal estradiol. The effect of increasing concentrations of glabridin on the growth of breast tumor cells was biphasic. Glabridin showed an estrogen receptor-dependent, growth-promoting effect at low concentrations (10 nM-10 μM) and estrogen receptor-independent antiproliferative activity at concentrations of >15 μM. This is the first study to indicate that isoflavans have estrogen-like activities. Glabridin and its derivatives exhibited varying degrees of estrogen receptor agonism in different tests and demonstrated growth-inhibitory actions on breast cancer cells (Tamir S., et al., Estrogenic and antiproliferative properties of glabridin from licorice in human breast cancer cells. Cancer Res. 2000; 60(20):5704-9).

D. Pueraria mirifica Extract and Miroestrol

Pueraria mirifica is an indigenous herb of Thailand, known in That as “Kwao Kreu” or “Kwao Kreu Kao” (White Kwao Kreu), It belongs to the Family Leguminosae, subfamily Papilionoideae or the soy, bean & pea subfamily. The plants are commonly found in abundant in the forests in the north, the west and the northeast of Thailand at the altitude of 300-800 meters above sea level. Active principles in this plant are found in the tuberous root, which looks like a chain of round-shaped bulbs of various sizes connected to the next one via small root throughout the entire length of the root. The shape and size of the tuberous root are diverse depending on the environment in which it exists.

Local communities in Thailand have used Pueraria mirifica for well over one hundred years, specifically for its rejuvenating qualities (Potee, Alicia. Health Sciences Institute. An ancient Thai “miracle” herb reveals itself to be a real-life fountain of youth. HSI, September 2007, Vol. 12, No. 3). The belief has been passing down from one generation to another and more recently through the publication by Luang Anusan Suntara, Women in the rural communities in Thailand where this herb grows have used the tuberous roots of Pueraria mirifica effectively as “rejuvenating” folk medicine for well over a hundred years. It has become well known and has received much attention from Thai and foreign scientists and mainstream alike not so many years ago. According to Thai traditional medicine, this “rejuvenating” herb is recommended for both aged men and women for its efficacy to grow hair, strengthen and darken existing ones, help improve complexion and remove wrinkles, improve eyesight, increase energy and vigor leading to more reflexive body movements.

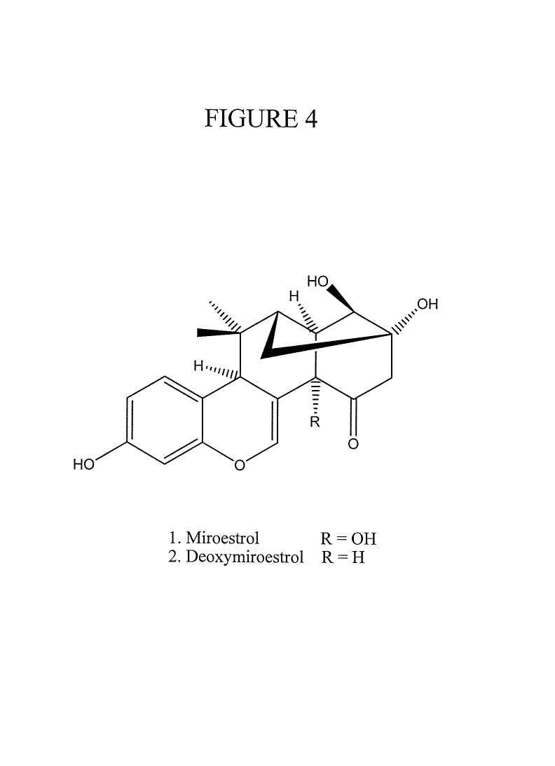

The compounds that make Pueraria mirifica different from any other phytoestrogen-containing plants in the Family Leguminosae are Miroestrol and Deoxymiroestrol (FIG. 4), which possess highest estrogenic activity among the known phytoestrogens due to structural similarity to estradiol (Chansakaow S., et al., Isoflavonoids from Pueraria Mirifica and their estrogenic activity. Planta Med 2000; 66:572-5).

The isolation and identification of deoxymiroestrol from the root of Pueraria mirifica has been reported in the February 2000 issue of the Journal of Natural Products. The authors proposed that since deoxymiroestrol is easily oxidized to miroestrol, deoxymiroestrol, not the previously reported Miroestrol, is more to be the actual chemical constituent of Pueraria mirifica. However, it is very likely that the two phytoestrogens coexist in the root of this plant. As shown below, the chemical structures of the two compounds are very similar to that of estradiol, the main human estrogen.

In addition to miroestrol and deoxymiroestrol, Pueraria mirifica also contains other chemicals that belong to isoflavone and coumestran groups of phytoestrogens, e.g., Genistein, Daidzein, Daidzin, Genistin, and Coumestrol that are usually found in soybeans (Table 2). However, the estrogenic activity of Miroestrol and Deoxymiroestrol is much more potent than that of soy isoflavones.

TABLE 2
Chromene, Isoflavonoid and Coumestan derivatives in Pueraria Mirifica

| | | | |
|---|---|---|---|
| Chromene | Isoflavones | Isoflavones | Coumestan |
| | | glycosides | |
| Miroestrol | Daidzein | Daidzin | Coumestrol |
| Deoxymiroestrol | Genistein | Genistin | Mirificoumestan |
| | Kwakhurin | Mirificin | Mirificoumestan |
| | | | glycol |
| | Kwakhurin | Puerarin | Mirificoumestan |
| | hydrate | | hydrate |
| | | Puerarin-6″ | |
| | | monoacetate | |

E. Tetrahydrocurcumin

Tetrahydrocurcumin (THC) is a colorless hydrogenated product derived from the yellow curcumin, the biologically active principle from the rhizomes of Curcuma longa (Turmeric), functions as efficient antioxidant compound. The superior antioxidant property of THC, combined with the lack of yellow color, render this product useful in achromatic food and cosmetic applications that currently employ conventional synthetic antioxidants (Majeed M., et al., (1995) Curcuminoids: Antioxidant Phytonutrients. Nutriscience Publishers, New Jersey). After absorption, curcumin is first biotransformed to dihydrocurcumin and THC and that these compounds subsequently are converted to monoglucuronide conjugates (Pan M., et al., (1999) Biotransformation of curcumin through reduction and glucuronidation in mice. Drug Metab. Dispos., 27(1):486-94).

Curcumin is reported to be potent antioxidant compounds by virtue of its molecular structure. THC has also shown significant antioxidant action in a number of in vitro and preclinical studies. Tetrahydrocurcumin is valued as the ultimate metabolites of the Curcuminoids in vivo. The poor circulating bioavailability of the parent curcuminoids, often attributed to their limited uptake due to poor water solubility, often impairs their biological effects in vivo (Huang, M., et al., (1997); Inhibitory effects of curcumin on tumorigenesis in mice. J. Cell. Biochem. Suppl., 27:26-34). Substantial beneficial effects could be achieved with lower levels of these active metabolites as compared to the parent compounds. Several independent studies reported the significant antioxidant effects of the tetrahydrocurcumin (Nakamura, Y., et al., (1998); Inhibitory effects of curcumin and tetrahydrocurcuminoids on the tumor promoter-induced reactive oxygen species generation in leukocytes, in vitro and in vivo. Jpn. J. Cancer Res., 89(4):361-70).

II. Pharmaceutical or Cosmetic Compositions

Certain embodiments of the present invention pertain to pharmaceutical or cosmetic compositions comprising the amyris alcohol or esters of amyris alcohol set forth herein. A variety of drug delivery systems may be used with the present invention, including topical and transdermal drug delivery systems.

The preparation of a pharmaceutical composition that contains at least one amyris alcohol or esterified amyris alcohol or additional active ingredient will be known to those of skill in the art in light of the present disclosure, as exemplified by Remington's Pharmaceutical Sciences, 18th Ed. Mack Printing Company, 1990, incorporated herein by reference.

Pharmaceutical or cosmetic compositions of the present invention will include an effective amount of amyris alcohol and/or one or more of the esters of amyris alcohol set forth herein useful in the treatment of acne. In some embodiments, the pharmaceutical or cosmetic compositions of the present invention further comprise an effective amount of glabridin, miroestrol, tetrahydrocurcumin or dihydroartemisinin and combinations thereof, including all of the listed agents. One of ordinary skill in the art would be familiar with what type of dosage is required for treatment of the particular pathological condition that is present in the subject. When used for therapy, the compositions of the present invention are administered to subjects in therapeutically effective amounts. For example, an effective amount of the amyris alcohol or ester of amyris alcohol in a patient with acne may be an amount that promotes the healing of the acne. The dose will depend on the nature of the disease, the subject, the subject's history, and other factors. Preparation of such compositions is discussed in other parts of this specification.

As discussed above, the derivatives set forth herein have greater lipophilicity and less irritation to the skin than amyris alcohols. One advantage of these esters is that they can be incorporated into a cream or ointment form at a higher percentage by weight as compared to amyris alcohol. Compositions employing the amyris alcohol or its ester set forth herein will contain a biologically effective amount of the ingredient. As used herein a biologically effective amount of an ingredient or composition refers to an amount effective to alter, modulate or reduce disease conditions. One of ordinary skill in the art would be familiar with methods of determining a biologically effective amount of a therapeutic agent. For example, in some embodiments, a biologically effective amount may be about 0.1 mg/kg to about 50 mg/kg or greater. For example, in some embodiments, a pharmaceutical or cosmetic composition to treat acne topically may contain from 1% to 90% by weight of amyris alcohol or the ester of amyris alcohol such as amyris acetate. In some embodiments, the amount of amyris alcohol or the ester of amyris alcohol is from 5% to 50% by weight, 5% and 20% by weight, or 10% to 20% by weight.

In some embodiments, the composition further comprises at least one of glabridin, miroestrol, tetrahydrocurcumin and dihydroartemisinin and combinations thereof. In some embodiments, the composition may contain from 0.1% to 20% by weight of tetrahydrocurcumin, from 0.5% to 5% by weight of tetrahydrocurcumin, or from 1% to 3% by weight of tetrahydrocurcumin. In some embodiments, the composition may contain from 0.1% to 5% by weight of glabridin, from 0.1% to 2% by weight of glabridin, or from 0.1% to 1% by weight of glabridin. In some embodiments, the composition may contain from 0.1 microgram per gram to 200 microgram per gram of miroestrol, from 1 microgram per gram to 50 microgram per gram of miroestrol, and from 3 microgram per gram to 30 microgram per gram of miroestrol. In some embodiments, the composition may contain from 0.1% to 5% by weight of dihydroartemisinin, from 0.5% to 2% by weight of dihydroartemisinin, and from 1% to 2% by weight of dihydroartemisinin.

The amyris alcohol or the ester of amyris alcohol such as amyris acetate of the present invention may be administered alone or as a mixture to treat acne. In other embodiments, the therapeutic amyris alcohol or amyris acetate is administered in combination with one or more secondary forms of therapy directed to the disease or condition to be treated. These are discussed in greater detail below. Additional pharmaceutical compounds may be administered in the same pharmaceutical or cosmetic composition, or in a separate dosage form, such as in a separate oral, intramuscular, or intravenous dosage forms taken at the same time.

The therapeutic or cosmetic agents of the present invention may be supplied in any form known to those of ordinary skill in the art. For example, the therapeutic agent may be supplied as a liquid or as a solution. The pharmaceutical or cosmetic compositions may contain a preservative to prevent the growth of microorganisms. It must be chemically and physically stable under the conditions of manufacture and storage and must be preserved against the contaminating action of microorganisms, such as bacteria and fungi. The prevention of the action of microorganisms can be brought about by various antibacterial and antifungal agents, for example, parabens, chlorobutanol, phenol, sorbic acid, thimerosal, and the like.

The formulations according to the invention having been described herein may influence the ordinarily skilled artesian to make similar formulations using components that will be known in the art, without departing from the invention which is claimed herein.

In some embodiments, the pharmaceutical or cosmetic formulations of the esters of amyris alcohol according to the present invention offer several advantages over the existing formulations. They can be topically applied and relatively high concentrations of the esters of amyris alcohol can be loaded into patients with high bioavailability. Thus the frequency of dosage can be reduced. Thus, in some embodiments, the invention provides improved formulations and methods of using the same when administering such formulations to patients. As mentioned herein above a number of excipients may be appropriate for use in the formulation. The inclusion of excipients and the optimization of their concentration for their characteristics such as for example ease of handling or carrier agents will be understood by those ordinarily skilled in the art not to depart from the spirit of the invention as described herein and claimed herein below.

Following preparation of the pharmaceutical or cosmetic compositions of the present invention, it may be desirable to quantify the amount of the esters of amyris alcohol in the pharmaceutical or cosmetic composition. Methods of measuring concentration of a drug in a composition include numerous techniques that are well-known to those of skill in the art. Selected examples include chromatographic techniques. There are many kinds of chromatography which may be used in the present invention: drug-specific assays, adsorption, partition, ion-exchange and molecular sieve, and many specialized techniques for using them including column, paper, thin-layer chromatography, gas chromatography, and high performance liquid chromatography (HPLC). One of ordinary skill in the art would be familiar with these and other related techniques.

III. Moisturizing Agents

Certain topical formulations of the present invention may contain moisturizing agents. Non-limiting examples of moisturizing agents that can be used with the compositions of the present invention include amino acids, chondroitin sulfate, diglycerin, erythritol, fructose, glucose, glycerin, glycerol polymers, glycol, 1,2,6-hexanetriol, honey, hyaluronic acid, hydrogenated honey, hydrogenated starch hydrolysate, inositol, lactitol, maltitol, maltose, mannitol, natural moisturization factor, PEG-15 butanediol, polyglyceryl sorbitol, salts of pyrollidone carboxylic acid, potassium PCA, propylene glycol, sodium glucuronate, sodium PCA, sorbitol, sucrose, trehalose, urea, and xylitol.

Other examples include acetylated lanolin, acetylated lanolin alcohol, acrylates/C10-30 alkyl acrylate crosspolymer, acrylates copolymer, alanine, algae extract, aloe barbadensis, aloe-barbadensis extract, aloe barbadensis gel, althea officinalis extract, aluminum starch octenylsuccinate, aluminum stearate, apricot (prunus armeniaca) kernel oil, arginine, arginine aspartate, arnica montana extract, ascorbic acid, ascorbyl palmitate, aspartic acid, avocado (persea gratissima) oil, barium sulfate, barrier sphingolipids, butyl alcohol, beeswax, behenyl alcohol, β-sitosterol, BHT, birch (betula alba) bark extract, borage (borago officinalis) extract, 2-bromo-2-nitropropane-1,3-diol, butcherbroom (ruscus aculeatus) extract, butylene glycol, calendula officinalis extract, calendula officinalis oil, candelilla (euphorbia cerifera) wax, canola oil, caprylic/capric triglyceride, cardamon (elettaria cardamomum) oil, carnauba (copernicia cerifera) wax, carrageenan (chondrus crispus), carrot (daucus carota sativa) oil, castor (ricinus communis) oil, ceramides, ceresin, ceteareth-5, ceteareth-12, ceteareth-20, cetearyl octanoate, ceteth-20, ceteth-24, cetyl acetate, cetyl octanoate, cetyl palmitate, chamomile (anthemis nobilis) oil, cholesterol, cholesterol esters, cholesteryl hydroxystearate, citric acid, clary (salvia sclarea) oil, cocoa (theobroma cacao) butter, coco-caprylate/caprate, coconut (cocas nucifera) oil, collagen, collagen amino acids, corn (zea mays) oil, fatty acids, decyl oleate, dextrin, diazolidinyl urea, dimethicone copolyol, dimethiconol, dioctyl adipate, dioctyl succinate, dipentaerythrityl hexacaprylate/hexacaprate, DMDM hydantoin, DNA, erythritol, ethoxydiglycol, ethyl linoleate, eucalyptus globulus oil, evening primrose (oenothera biennis) oil, fatty acids, fructose, gelatin, geranium maculatum oil, glucosamine, glucose glutamate, glutamic acid, glycereth-26, glycerin, glycerol, glyceryl distearate, glyceryl hydroxystearate, glyceryl laurate, glyceryl linoleate, glyceryl myristate, glyceryl oleate, glyceryl stearate, glyceryl stearate SE, glycine, glycol stearate, glycol stearate SE, glycosaminoglycans, grape (vitis vinifera) seed oil, hazel (corylus americana) nut oil, hazel (corylus avellana) nut oil, hexylene glycol, honey, hyaluronic acid, hybrid safflower (carthamus tinctorius) oil, hydrogenated castor oil, hydrogenated coca-glycerides, hydrogenated coconut oil, hydrogenated lanolin, hydrogenated lecithin, hydrogenated palm glyceride, hydrogenated palm kernel oil, hydrogenated soybean oil, hydrogenated tallow glyceride, hydrogenated vegetable oil, hydrolyzed collagen, hydrolyzed elastin, hydrolyzed glycosaminoglycans, hydrolyzed keratin, hydrolyzed soy protein, hydroxylated lanolin, hydroxyproline, imidazolidinyl urea, iodopropynyl butylcarbamate, isocetyl stearate, isocetyl stearoyl stearate, isodecyl oleate, isopropyl isostearate, isopropyl lanolate, isopropyl myristate, isopropyl palmitate, isopropyl stearate, isostearamide DEA, isostearic acid, isostearyl lactate, isostearyl neopentanoate, jasmine (jasminum officinale) oil, jojoba (buxus chinensis) oil, kelp, kukui (aleurites moluccana) nut oil, lactamide MEA, laneth-16, laneth-10 acetate, lanolin, lanolin acid, lanolin alcohol, lanolin oil, lanolin wax, lavender (lavandula angustifolia) oil, lecithin, lemon (citrus medica limonum) oil, linoleic acid, linolenic acid, macadamia ternifolia nut oil, magnesium stearate, magnesium sulfate, maltitol, matricaria (chamomilla recutita) oil, methyl glucose sesquistearate, methylsilanol PCA, microcrystalline wax, mineral oil, mink oil, mortierella oil, myristyl lactate, myristyl myristate, myristyl propionate, neopentyl glycol dicaprylate/dicaprate, octyldodecanol, octyldodecyl myristate, octyldodecyl stearoyl stearate, octyl hydroxystearate, octyl palmitate, octyl salicylate, octyl stearate, oleic acid, olive (olea europaea) oil, orange (citrus aurantium dulcis) oil, palm (elaeis guineensis) oil, palmitic acid, pantethine, panthenol, panthenyl ethyl ether, paraffin, PCA, peach (prunus persica) kernel oil, peanut (arachis hypogaea) oil, PEG-8 C12-18 ester, PEG-15 cocamine, PEG-150 distearate, PEG-60 glyceryl isostearate, PEG-5 glyceryl stearate, PEG-30 glyceryl stearate, PEG-7 hydrogenated castor oil, PEG-40 hydrogenated castor oil, PEG-60 hydrogenated castor oil, PEG-20 methyl glucose sesquistearate, PEG40 sorbitan peroleate, PEG-5 soy sterol, PEG-10 soy sterol, PEG-2 stearate, PEG-8 stearate, PEG-20 stearate, PEG-32 stearate, PEG40 stearate, PEG-50 stearate, PEG-100 stearate, PEG-150 stearate, pentadecalactone, peppermint (mentha piperita) oil, petrolatum, phospholipids, polyamino sugar condensate, polyglyceryl-3 diisostearate, polyquaternium-24, polysorbate 20, polysorbate 40, polysorbate 60, polysorbate 80, polysorbate 85, potassium myristate, potassium palmitate, potassium sorbate, potassium stearate, propylene glycol, propylene glycol dicaprylate/dicaprate, propylene glycol dioctanoate, propylene glycol dipelargonate, propylene glycol laurate, propylene glycol stearate, propylene glycol stearate SE, PVP, pyridoxine dipalmitate, quaternium-15, quaternium-18 hectorite, quaternium-22, retinol, retinyl palmitate, rice (oryza sativa) bran oil, RNA, rosemary (rosmarinus officinalis) oil, rose oil, safflower (carthamus tinctorius) oil, sage (salvia officinalis) oil, salicylic acid, serine, serum protein, sesame (sesamum indicum) oil, shea butter (butyrospermum parkii), silk powder, sodium chondroitin sulfate, sodium DNA, sodium hyaluronate, sodium lactate, sodium palmitate, sodium PCA, sodium polyglutamate, sodium stearate, soluble collagen, sorbic acid, sorbitan laurate, sorbitan oleate, sorbitan palmitate, sorbitan sesquioleate, sorbitan stearate, sorbitol, soybean (glycine soja) oil, sphingolipids, squalane, squalene, stearamide MEA-stearate, stearic acid, stearoxy dimethicone, stearoxytrimethylsilane, stearyl alcohol, stearyl glycyrrhetinate, stearyl heptanoate, stearyl stearate, sunflower (helianthus annuus) seed oil, sweet almond (prunus amygdalus dulcis) oil, synthetic beeswax, tocopherol, tocopheryl acetate, tocopheryl linoleate, tribehenin, tridecyl neopentanoate, tridecyl stearate, triethanolamine, tristearin, urea, vegetable oil, water, waxes, wheat (triticum vulgare) germ oil, and ylang ylang (cananga odorata) oil.

IV. Antioxidants

Certain topical formulations of the present invention may also contain one or more antioxidants in addition to tetrahydrocurcumin. Non-limiting examples of antioxidants that can be used with the compositions of the present invention include acetyl cysteine, ascorbic acid, ascorbic acid polypeptide, ascorbyl dipalmitate, ascorbyl methylsilanol pectinate, ascorbyl palmitate, ascorbyl stearate, BHA, BHT, t-butyl hydroquinone, cysteine, cysteine HCl, diamylhydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, hydroquinone, isooctyl thioglycolate, kojic acid, magnesium ascorbate, magnesium ascorbyl phosphate, methylsilanol ascorbate, natural botanical anti-oxidants such as green tea or grape seed extracts, nordihydroguaiaretic acid, octyl gallate, phenylthioglycolic acid, potassium ascorbyl tocopheryl phosphate, potassium sulfite, propyl gallate, quinones, rosmarinic acid, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, resveratrol and tris(nonylphenyl)phosphite.

V. Secondary Therapies

Some embodiments of the claimed methods of the present invention involve administering to the subject a secondary form of therapy in addition to amyris alcohol or amyris acetate set forth herein. If the disease is acne, exemplary secondary forms of therapy include systemic antibiotics and steroids.

In order to increase the effectiveness of the therapeutic agent disclosed herein, it may be desirable to combine the therapeutic agent of the present invention with the secondary therapeutic agent. These compositions would be provided in a combined amount effective to provide for a therapeutic response in a subject. One of ordinary skill in the art would be able to determine whether the subject demonstrated a therapeutic response. This process may involve administering the therapeutic agent of the present invention and the secondary therapeutic agent to the subject at the same time. This may be achieved by administering a single composition or pharmacological formulation that includes both agents, or by administering two distinct compositions or formulations, at the same time, wherein one composition includes the curcumin derivative of the present invention and the other includes the secondary agent.

Alternatively, the therapeutic agent of the present invention may precede or follow the treatment with the secondary agent by intervals ranging from minutes to weeks. In embodiments where the secondary agent and the amyris alcohol or amyris acetate of the present invention are separately administered, one would generally ensure that a significant period of time did not expire between the time of each delivery, such that the secondary agent and the therapeutic agent of the present invention would still be able to exert a beneficial effect on the subject. In such instances, it is contemplated that one may administer both modalities within about 24-48 h of each other and, more preferably, within about 12-24 h of each other, and even more preferably within about 30 minute-6 h of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

Various combinations may be employed, the therapeutic agent of the present invention is “A” and the secondary agent, such as antibiotic or steroid therapy, is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of the compositions of the present invention to a patient will follow general protocols for the administration of therapeutic agents. It is expected that the treatment cycles would be repeated as necessary.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLES

### Example 1

### Preparation of Acetyl Ester of Amyris Alcohol (Formula Ia, R=CH₃)

A mixture of 100 ml (˜0.4M of alcohol content) of amyris alcohol (Texarome Inc, Leakey, Tex.), 190 ml (2M) of acetic anhydride and 5 drops of H₃PO₄ (85% in water) were introduced in a 1000 ml flask, and the mixture was stirred over night at reflux condition. Afterwards, 2 L of water where added and the stirring was prolonged for an additional period of 2 hours. The crude product was extracted by washing the water solution with 1 L of n-hexane. The organic phase thus obtained were washed twice with a saturated NaHCO₃ water solution, then twice with brine and finally dried over anhydrous MgSO₄ and concentrated. It was thus obtained 130 g of crude product (95% yield) having a GC purity of >75%.

### Example 2

### Preparation of Topical Gel Containing Amyris Alcohol

The following procedure was used to prepare a 10% gel containing amyris alcohol.
Batch size: 2.0 kg

### Preparation of Base Gel, Part A

In a clean pail, add and mix using mechanical mixer,

| | | |
|---|---|---|
| Purified water, hot | 1000 | ml |
| Potassium sorbate | 1.0 | g (0.05%) |
| Methyl paraben sodium | 2.0 | g (0.10%) |
| Propyl paraben sodium | 1.0 | g (0.05%) |
| Disodium edetate | 10.0 | g (0.50%) |
| Benzalkonium chloride | 2.0 | g (0.10%) |
| Add and mix, | | |
| Carbopol Ultrez10 | 60.0 | g (3.0%) |
| Xanthan gum | 6.0 | g (0.3%) |

This is the base gel mixture and set aside.

### Preparation of Organic Phase, Part B

In another container, add the following and heat to 60-70 degrees C. to dissolve,

| | |
|---|---|
| Tween 80 | 12.0 g (0.6%) |
| Hallbrite BHB | 60.0 g (3.0%) |
| Eugenyl aceteate | 20.0 g (1.0%) |
| Cetyl myristoleate | 40.0 g (2.0%) |
| Isobornyl propionate | 10.0 g (0.5%) |
| Amyris alcohol | 200.0 g (10.0%) |
| Glycerin | 100.0 g (5.0%) |

Now to the above base gel, using high speed mixer, add and mix Part B until smooth gel is formed.

| Then add and mix, | | |
|---|---|---|
| Triethanolamine, 99% | 32.0 | g (1.6%) |
| Lavender oil | 16.0 | ml (0.8%) |
| Water, purified to make QS | 2000 | g |

Homogenize for 5 min. using high speed Waring homogenizer.

The pH of this gel is between 5.5 and 6.5 and the gel is obtained as a smooth, shiny and good texture cream.

In a similar manner, the gel containing amyris acetate was prepared by replacing amyris alcohol with amyris acetate.

### Example 3

### Preparation of Topical Gel Containing Amyris Alcohol, Phytoestrogens, Peroxide and Antioxidant

The following procedure was used to prepare a 10% gel containing amyris alcohol.
Batch size: 2.0 kg

### Preparation of Base Gel, Part A

In a clean pail, add and mix using mechanical mixer,

| | | |
|---|---|---|
| Purified water, hot | 1000 | ml |
| Potassium sorbate | 1.0 | g (0.05%) |
| Methyl paraben sodium | 2.0 | g (0.10%) |
| Propyl paraben sodium | 1.0 | g (0.05%) |
| Disodium edetate | 10.0 | g (0.50%) |
| Benzalkonium chloride | 2.0 | g (0.10%) |
| Add and mix, | | |
| Carbopol Ultrez10 | 60.0 | g (3.0%) |
| Xanthan gum | 6.0 | g (0.3%) |

This is the base gel mixture and set it aside.

### Preparation of Organic Phase, Part B

In another container, add the following and heat to 60-70 degrees C. to dissolve,

| | |
|---|---|
| Tween 80 | 12.0 g (0.6%) |
| Hallbrite BHB | 60.0 g (3.0%) |
| Eugenyl aceteate | 20.0 g (1.0%) |
| Cetyl myristoleate | 40.0 g (2.0%) |
| Isobornyl propionate | 10.0 g (0.5%) |
| Amyris alcohol | 200.0 g (10.0%) |

Keep this hot. This is Part #1

In another container, add and heat to 60-70 degrees C. to dissolve

| | |
|---|---|
| Dihydro tetrahydrocurcumin | 40.0 g (2.0%) |
| Dihydroartemisinin | 20.0 g (1.0%) |
| Glabridine | 4.0 g (0.2%) |
| Propylene glycol | 100.0 g (5.0%) |

Keep this hot. This is part #2.

In another container, add and dissolve

| | | |
|---|---|---|
| Puereria Mirifica Extract | 20.0 | g (1.0%) |
| Purified water | 100 | ml |
| Glycerin | 100.0 | g (5.0%) |

Keep it aside and this is part #3.

Now to the above base gel, using high speed mixer, add and mix part #1, part #2 and part #3 in succession until smooth gel is formed.

| Then add and mix, | | |
|---|---|---|
| Triethanolamine, 99% | 32.0 | g (1.6%) |
| Lavender oil | 16.0 | ml (0.8%) |
| Water, purified to make QS | 2000 | g |

Homogenize for 5 min. using high speed Waring homogenizer.

The pH of this gel is between 5.5 and 6.5 and the gel is obtained as a smooth, shiny and good texture cream.

### Example 4

### Toxicity Assessment of the Inventive Composition

A 10% topical gel of amyris alcohol as described in example 3, was applied to the forearm of 12 healthy individuals twice daily for a two-week period in an outpatient clinic. No patients complained of burning, irritation, scaling or redness after the cream ws applied. Patients returned to the clinic after having used the solution for two weeks for a visual inspection of the forearm area. The examining physician noted no redness, irritation or scaling in the area where the solution had been applied.

### Example 5

A 40 year old white female develops acne periodically on her face. She was given the 10% topical gel of amyris alcohol described in Example 3, code named Dermazol, and the following is her testimony on the effectiveness of the gel for the treatment of acne. I have been using Dermazol for approximately 2 months for mild acne and have experienced great results. At the onset of a pimple when I first see redness, I apply Dermazol after cleaning my face. Within a short amount of time the redness is gone. Other times, after seeing a pimple starting to protrude the skin I apply Dermazol a few times during the day and by the following day it has completely disappeared. I've never had a pimple last more than a day since using Dermazol. Dermazol doesn't dry my skin and I love the creamy texture—its like applying a luxury face cream!”

We claim:

1. A method of treating acne in a human in need of treatment, said method comprising topically administering to the human a pharmaceutical or cosmetic composition comprising an effective amount of amyris alcohol or an ester of amyris alcohol or a mixture thereof.

2. The method of claim 1, wherein the ester of amyris alcohol is selected from the group consisting of amyris acetate, amyris propionate, amyris butanoate, amyris palmitate and amyris stearate.

3. The method of claim 1, wherein the pharmaceutical or cosmetic composition further comprises at least one of glabridin, miroestrol, tetrahydrocurcumin and dihydroartemisinin.

4. The method of claim 1, wherein the pharmaceutical or cosmetic composition is provided in a topical formulation.

5. The method of claim 1, wherein the topical formulation is selected from the group consisting of a cream, lotion, spray and wipe formulation.

6. The method of claim 1, wherein the pharmaceutical or cosmetic composition comprises an amount of amyris alcohol or the ester of amyris alcohol selected from the group consisting of from 1% to 90% by weight of the amyris alcohol or the ester of amyris alcohol; from 5% to 50% by weight of amyris alcohol or the ester of amyris alcohol; and from 10% to 20% by weight of amyris alcohol or the ester of amyris alcohol.

7. The method of claim 3, wherein the pharmaceutical or cosmetic composition comprises an amount of glabridin selected from the group consisting of from 0.1% to 5% by weight of glabridin; from 0.1% to 2% by weight of glabridin; and from 0.1% to 1% by weight of glabridin.

8. The method of claim 3, wherein the pharmaceutical or cosmetic composition comprises an amount of miroestrol selected from the group consisting of from 0.1 microgram per gram to 200 microgram per gram of miroestrol; from 1 microgram per gram to 50 microgram per gram of miroestrol; and from 3 microgram per gram to 30 microgram per gram of miroestrol.

9. The method of claim 3, wherein the pharmaceutical or cosmetic composition comprises an amount of tetrahydrocurcumin selected from the group consisting of from 0.1% to 20% by weight of tetrahydrocurcumin; from 0.5% to 5% by weight of tetrahydrocurcumin; and from 1% to 3% by weight of tetrahydrocurcumin.

10. The method of claim 3, wherein the pharmaceutical or cosmetic composition comprises an amount of dihydroartemisinin selected from the group consisting of from 0.1% to 5% by weight of dihydroartemisinin; from 0.5% to 2% by weight of dihydroartemisinin; and from 1% to 2% by weight of dihydroartemisinin.

11. A pharmaceutical or cosmetic composition comprising from 1% to 90% by weight of amyris alcohol or an ester of amyris alcohol, and an additional agent selected from the group consisting of from 0.1% to 5% by weight of glabridin, from 0.1 microgram per gram to 200 microgram per gram of miroestrol, from 0.1% to 20% by weight of tetrahydrocurcumin and from 0.1% to 5% by weight of dihydroartemisinin and combinations thereof.
